(19) United States
(12) Patent Application Publication
(10) Pub. No.: US 2026/0185057 A1
(43) Pub. Date: Jul. 2, 2026

(54) BIOCOMPATIBILITY OF ZINC PHOSPHATE NANOPARTICLES AND METHODS OF PREPARATION THEREOF

(71) Applicant: Imam Abdulrahman Bin Faisal University, Dammam (SA)

(72) Inventors: Ebtesam A. AL-SUHAIMI, Dammam (SA); Noha BIN MAHFOOZ, Dammam (SA); Abdelgadir Musa HOMEIDA, Dammam (SA); Muhammad NAWAZ, Dammam (SA)

(73) Assignee: Imam Abdulrahman Bin Faisal University, Dammam (SA)

(21) Appl. No.: 19/262,821

(22) Filed: Jul. 8, 2025

Related U.S. Application Data
(60) Provisional application No. 63/740,156, filed on Dec. 30, 2024.

(51) Int. Cl. C12N 5/00

(52) U.S. Cl. CPC C12N 5/522

(57) ABSTRACT

A method for reducing hemolysis induced by dimethyl sulfoxide (DMSO) includes contacting a sample including red blood cells (RBCs) with an organic solvent composition including DMSO and zinc phosphate nanoparticles (ZnPNPs). The ZnPNPs are crystalline and are in the form of agglomerated, overlapping asymmetrical semi-spherical particles with an average size of 30 to 60 nanometers (nm). The ZnPNPs are in the form of rectangular flakes. The ZnPNPs include 30 to 35 wt. % oxygen (O), 12 to 18 wt. % phosphorous (P), and 50 to 55 wt. % zinc (Zn). The sample including RBCs contacted with the organic solvent composition provides a reduction in hemolysis of the red blood cells 4 to 5 times that of a DMSO-containing composition that is the same as the organic solvent composition but does not include the ZnPNPs.

### CROSS REFERENCE TO RELATED APPLICATION

This application claims the benefit of U.S. Provisional Application No. 63/740,156, filed Dec. 30, 2024, which is incorporated herein by reference in its entirety.

### BACKGROUND

### Technical Field

The present disclosure is directed to a method of reducing hemolysis, and more particularly, a method of reducing hemolysis induced by dimethyl sulfoxide (DMSO) in red blood cells (RBCs) using zinc phosphate nanoparticles (ZnNPs).

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present disclosure.

Advancements in the biomaterials industry have made medical materials useful in clinical repairs of damage to human tissue; however, in recent decades, environmental pollution caused by pathogenic bacteria has become a threat to human health, posing challenges for sterilization and bacteriostasis research. Hemolysis, the rupture or destruction of red blood cells (RBCs), has emerged as a concern in medical and biotechnological applications. Hemolysis can result from various factors, including exposure to organic solvents such as dimethyl sulfoxide (DMSO), mechanical stress, and/or toxic agents. This condition not only disrupts physiological functions but also causes severe complications, such as anemia, oxidative stress, and kidney damage. Together, these challenges highlight the need for innovative solutions to address bacterial contamination and hemolysis effectively.

Traditional methods to mitigate hemolysis have focused on varying solvent concentrations, modifying procedural protocols, and/or incorporating protective additives. Despite these efforts, achieving a balance between safety and efficacy has remained a challenge. Recent advancements in nanotechnology have provided alternatives such as using nanoparticles (NPs) in conjunction with organic solvents. NPs, characterized by their crystalline structure, offer high surface area and biocompatibility and make them highly effective in stabilizing RBCs and reducing hemolytic effects. Biomedical metal materials, including those made of titanium and titanium alloys, have been widely used in artificial joints, dental implants, orthopedic prostheses and other fields. Biomedical polymer materials have also been widely used; however, in clinical applications, medical materials still face problems of bacterial infection.

By interacting with solvents like DMSO, NPs help preserve the integrity of RBC membranes, minimizing damage and enhancing cell stability. Among these, nanoparticles containing zinc have shown promise over their properties their micron-sized counterparts. With higher surface potential energy, a larger specific surface area, and increased surface grain boundaries, nanoparticles containing zinc release more zinc ions and improve material-bacteria interactions. NPs not only boosts their antibacterial efficacy but also highlights their versatility as a solution for addressing bacterial contamination and hemolysis-related challenges in medical and industrial applications, promoting better patient outcomes and advancing biomedical research.

Accordingly, an object of the present disclosure is to provide a method of reducing hemolysis induced by DMSO using zinc phosphate NPs (ZnPNPs), that may circumvent the drawbacks and limitations, such as the potential toxicity of organic solvents at higher concentrations, the limited efficacy of conventional additives in preventing RBC rupture, and the inability to maintain RBC integrity under prolonged exposure or under mechanical stress, of the methods and materials known in the art.

### SUMMARY

In an exemplary embodiment, a method of reducing hemolysis induced by dimethyl sulfoxide (DMSO) is described. The method includes contacting a sample including red blood cells (RBCs) with an organic solvent composition including DMSO and zinc phosphate nanoparticles (ZnPNPs). The ZnPNPs are crystalline. The ZnPNPs are in the form of agglomerated, overlapping asymmetrical semi-spherical particles with an average size of 30 to 60 nanometers (nm). The ZnPNPs are in the form of rectangular flakes. The ZnPNPs include 30 to 35 precent by weight (wt. %) oxygen, 12 to 18 wt. % phosphorous, and 50 to 55 wt. % zinc. The sample including RBCs contacted with the organic solvent composition provides a reduction in hemolysis of the red blood cells that is 4 to 5 times that of a DMSO-containing composition that is the same as the organic solvent composition but does not include the ZnPNPs.

In some embodiments, the concentration of the ZnPNPs in the organic solvent composition is 0.5 to 10 milligrams per milliliter (mg/mL).

In some embodiments, the concentration of the DMSO in the organic solvent composition is 0.1 to 1 percent by volume (vol. %) based on a total volume of the organic solvent composition.

In some embodiments, the sample including RBCs has a free hemoglobin level of 0.05 to 0.25 grams per liter (g/L) after the contacting.

In some embodiments, the ZnPNPs create a barrier on a surface of the RBCs.

In some embodiments, the zinc phosphate nanoparticle barrier on the surface of the RBCs does not allow the DMSO to penetrate the RBCs.

In some embodiments, 75% to 95% of the RBCs in the sample are viable after the contacting.

In some embodiments, the method further includes sonicating the ZnPNPs in a polar solvent to form a solution and contacting the solution with test cells. At least 15% of the test cells are viable at a zinc phosphate nanoparticle concentration of 1 mg/mL or less.

In some embodiments, the test cells are human embryonic kidney (HEK 293) cells.

In some embodiments, 105% to 115% of the test cells are viable at a solution concentration of 0.05 to 0.08 mg/mL.

In some embodiments, 110% to 115% of the test cells are viable at a solution concentration of 0.1 to 0.15 mg/mL.

In some embodiments, 100% to 110% of the test cells are viable at a solution concentration of 0.2 to 0.3 mg/mL.

In some embodiments, the ZnPNPs have a median lethal dose (LD₅₀) in rats of 2400 to 2600 milligrams per kilogram (mg/kg) of body weight.

In some embodiments, the ZnPNPs have an LD₅₀ in mice of 4800 to 5200 mg/k of body weight.

In some embodiments, the contacting is from 5 to 50 days.

In some embodiments, the polar solvent includes water, DMSO, and a phosphate-buffered saline (PBS).

In some embodiments, the ZnPNPs have a polydispersity index of 0.8 to 0.9.

In some embodiments, the ZnPNPs have a zeta potential of −40 to −30 millivolts (mV).

In some embodiments, the ZnPNPs include 33 to 34 wt. % oxygen, 14 to 15 wt. % phosphorous, and 52 to 53 wt. % zinc based on a total weight of the ZnPNPs.

In some embodiments, a method of making the ZnPNPs is described. The method includes dissolving a zinc salt in water and sonicating for 1 to 10 minutes (min) to form a first solution and dissolving a phosphate salt in water and sonicating for 1 to 20 min to form a second solution. The method includes adding the second solution to the first solution to form a first mixture including particles and a liquid, sonicating the first mixture for 20 to 60 min, centrifuging the first mixture for 1 to 10 min, and decanting the liquid from the first mixture to separate the particles from the liquid. The method further includes washing the particles with water and centrifuging, washing the particles with a polar solvent, drying the particles, calcinating the particles at a temperature of 700 to 900 degrees Celsius (° C.) for 1 to 5 hours (h) to form calcinated particles, and crushing the calcinated particles to form the ZnPNPs.

The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 illustrates a method flow chart for forming zinc phosphate nanoparticles (ZnPNPs), according to certain embodiments.

FIG. 2 is a schematic diagram depicting synthesis steps of the ZnPNPs from zinc nitrate (Zn(NO₃)₂) and sodium phosphate (Na₃PO₄), according to certain embodiments.

FIG. 3 is an X-ray diffraction (XRD) pattern of the ZnPNPs, according to certain embodiments.

FIG. 4A is a transmission electron microscopy (TEM) image of the ZnPNPs with a magnification scale of 100 nanometers (nm), according to certain embodiments.

FIG. 4B is a TEM image of the ZnPNPs with a magnification scale of 100 nm, according to certain embodiments.

FIG. 5A is a scanning electron microscopy (SEM) image of the ZnPNPs, according to certain embodiments.

FIG. 5B is an SEM image of an area with a flake-like structure of the ZnPNPs, according to certain embodiments.

FIG. 5C is an SEM image of the ZnPNPs, according to certain embodiments.

FIG. 6A is an energy-dispersive X-ray (EDX) spectra of the ZnPNPs, according to certain embodiments.

FIG. 6B is an elemental mapping image depicting the distribution of zinc (Zn) in the ZnPNPs, according to certain embodiments.

FIG. 6C is an elemental mapping image depicting the distribution of phosphorous (P) in the ZnPNPs, according to certain embodiments.

FIG. 6D is an elemental mapping image depicting the distribution of oxygen (O) in the ZnPNPs, according to certain embodiments.

FIG. 7A is a graph depicting particle size distribution of the ZnPNPs, according to certain embodiments.

FIG. 7B is a graph depicting zeta potential of the ZnPNPs, according to certain embodiments.

FIG. 8 is a schematic illustration of a hemolysis test protocol using microtubes containing rat red blood cells (RBCs) treated with the ZnPNPs, according to certain embodiments.

FIG. 9 depicts microtubes containing rat RBCs treated with the ZnPNPs, according to certain embodiments.

FIG. 10A is a bar graph of cell viability (%) in HEK293 treated with a 5-fluorouracil drug, according to certain embodiments.

FIG. 10B depicts cell viability (%) in HEK293 treated with the ZnPNPs, according to certain embodiments.

FIG. 11 is a graph depicting a hemolysis percentage (%) in HEK293 treated with the ZnPNPs, according to certain embodiments.

FIG. 12A is a plot depicting median lethal dose (LD₅₀) of the ZnPNPs in mice, according to certain embodiments.

FIG. 12B is a plot depicting LD₅₀ of the ZnPNPs in rats, according to certain embodiments.

### DETAILED DESCRIPTION

When describing the present disclosure, the terms used are to be construed in accordance with the following definitions, unless a context dictates otherwise.

Embodiments of the present invention will now be described more fully hereinafter with reference to the accompanying drawings wherever applicable, in that some, but not all, embodiments of the disclosure are shown.

In the drawings, like reference numerals will be used to designate identical or corresponding parts throughout the several views. Further, as used herein, the words “a,” “an,” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

When amounts, concentrations, dimensions and other parameters are expressed in the form of a range, a preferable range, an upper limit value, a lower limit value, or preferable upper and limit values, it should be understood that any ranges obtainable by combining any upper limit or preferable value with any lower limit or preferable value are also specifically disclosed, irrespective of whether the obtained ranges are clearly mentioned in the context.

As used herein, the term “amount” refers to the level or concentration of one or more reactants, catalysts, and/or materials present in a reaction mixture.

As used herein, the term “compound” refers to a chemical entity, regardless of its phase—solid, liquid, or gaseous—as well as its state—crude mixture, purified, or isolated.

As used herein, the term “particle” refers to an object that acts as a whole unit with regard to its transport and properties.

As used herein, the term “nanoparticles (NPs)” refers to an object or material (i.e., particles) having a particle size of 1 to 500 nanometers (nm).

As used herein, the term “nanocomposite” refers to a material comprising various parts or elements that has at least one component with a grain size measured in nanometers.

As used herein, the term “particle size” may be thought of as a length or the longest dimension of a particle. The longest distance that can be measured from one point on a shape through its center to a point directly across from it is referred to as the “diameter” for a circle, oval, ellipse, and multilobe.

As used herein, the term “organic” refers to a substance or material that can be produced from naturally occurring substances or materials.

As used herein, the term “room temperature” refers to a temperature range of 23±3° C. degrees Celsius (° C.) in the present disclosure.

As used herein, the term “ambient conditions” refers to the surroundings in which the substance, composition or article is located and may include temperature, pressure, humidity, air pressure, light intensity, noise level, and vibration.

As used herein, the term “deionized water (DW)” refers to the water that has (most of) the ions removed.

As used herein, the term “red blood cells (RBCs)” refers to a type of blood cell used for transporting oxygen from the lungs to the tissues and organs of the body and carrying carbon dioxide back to the lungs for exhalation. These cells contain the protein hemoglobin, which binds oxygen, and are characterized by their biconcave shape and lack of a nucleus in mammals.

As used herein, the term “hemolysis” refers to the rupture or destruction of RBCs, resulting in the release of hemoglobin and other intracellular components into the surrounding fluid or plasma.

As used herein, the term “polydispersity index (PDI)” refers to a measure of the width of distribution of an average particle size within a sample. As used herein, PDI is measured via dynamic light scattering. PDI is a dimensionless value that quantifies the uniformity of the sample, with lower values indicating a more uniform distribution and higher values representing greater heterogeneity.

As used herein, the term “Z-average” refers to an intensity-weighted mean hydrodynamic size on an ensemble collection of particles measured by dynamic light scattering (DLS). In DLS, the Z-average is used to present the average particle size, which is the intensity-weighted average size. PDI indicates a width of the distribution of the average particle size.

As used herein, the term “zeta potential” refers to a measure of an electrical potential at a shear plane of a particle in a suspension. It indicates the degree of electrostatic repulsion or attraction between particles, which affects the stability of colloidal dispersions. A higher absolute value of zeta potential signifies better dispersion stability, as it helps prevent particle aggregation.

As used herein, the term “sonication” refers to a process in which sound waves are used to agitate particles in a solution.

As used herein, the term “toxicity” refers to harmful effects of a substance on living organisms, potentially causing injury or disruption of normal physiological functions.

As used herein, the term “cytotoxicity” refers to an ability of a substance or process to damage or kill cells. The word “cyto” means cell and “toxic” means poison.

As used herein, the term “median lethal dose (LD₅₀)” refers to a dose of a substance that causes death in 50% of a test population under specified conditions. It is used in toxicology to evaluate acute toxicity of a substance and is typically expressed in units such as milligrams of substance per kilogram of body weight (mg/kg).

A weight percent of a component, unless specifically stated to the contrary, is based on the total weight of the formulation or composition in which the component is included. For example, if a particular element or component in a composition or article is said to have 5 wt. %, it is understood that this percentage is in relation to a total compositional percentage of 100%.

The present disclosure is intended to include all hydration states of a given compound or formula, unless otherwise noted or when heating a material.

In addition, the present disclosure is intended to include all isotopes of atoms occurring in the present compounds and complexes. Isotopes include those atoms having the same atomic number but different mass numbers. By way of general example, and without limitation, isotopes of hydrogen include deuterium and tritium, and isotopes of carbon include ¹³C and ¹⁴C. Isotopes of oxygen include ¹⁶O, ¹⁷O, and ¹⁸O. Isotopically-labeled compounds of the present disclosure may generally be prepared by conventional techniques known to those skilled in the art or by processes analogous to those described herein, using an appropriate isotopically-labeled reagent in place of the non-labeled reagent otherwise employed.

Aspects of this disclosure are directed to a method of reducing hemolysis induced by dimethyl sulfoxide (DMSO) in red blood cells (RBCs) using zinc phosphate nanoparticles (ZnPNPs). This improves viability and integrity of cells, enhancing the effectiveness of treatments.

A method of reducing hemolysis induced by dimethyl sulfoxide (DMSO) is described. The method includes contacting a sample including red blood cells (RBCs) with an organic solvent including DMSO and zinc phosphate nanoparticles (ZnPNPs). An organic solvent is a carbon-based substance employed for the dissolution of one or more substances. In other embodiments, organic solvents including, but not limited to, ethanol, methanol, propanol, isopropanol (IPA), butanol, acetone, ethyl acetate, benzene, toluene, xylene, chloroform, dichloromethane, tetrahydrofuran (THF), dimethylformamide (DMF), acetonitrile, hexane, heptane, octane, nonane, decane, dioxane, diethyl ether, petroleum ether, naphtha, formamide, pyridine, nitrobenzene, anisole ethylene glycol, a combination thereof, and the like may be used in combination with or in place of the DMSO.

In some embodiments, the concentration of the ZnPNPs is 0.5-10 milligrams per milliliter (mg/mL), preferably 1-9 mg/mL, preferably 2-8 mg/mL, preferably 3-7 mg/mL, preferably 4-6 mg/mL, and preferably about 5 mg/mL. In a preferred embodiment, the concentration of the ZnPNPs is about 1 mg/mL.

In some embodiments, the concentration of DMSO is 0.1-1 percent by volume (vol. %), preferably 0.2-0.9 vol. %, preferably 0.3-0.8 vol. %, preferably 0.4-0.7 vol. %, more preferably 0.5-0.6 vol. %, and yet more preferably about 0.5 vol. %, based on a total volume of the organic solvent.

The ZnPNPs are crystalline. In some embodiments, the ZnPNPs may include crystalline phases including, but not limited to, quartz, calcite, hematite, magnetite, goethite, dolomite, albite, anorthite, pyrite, fluorite, halite, barite, apatite, rutile, zircon, a combination thereof, and the like.

The ZnPNPs are in the form of agglomerated, overlapping asymmetrical semi-spherical particles with an average size of 30-60 nm, preferably 31-59 nm, preferably 32-58 nm, preferably 33-57 nm, preferably 34-56 nm, preferably 35-55 nm, preferably 36-54 nm, preferably 37-53 nm, preferably 38-52 nm, preferably 39-51 nm, preferably 40-50 nm, preferably 41-49 nm, preferably 42-48 nm, preferably 43-47 nm, and preferably 44-46 nm. In a preferred embodiment, the average size of the ZnPNPs is about 43 nm. In some embodiments, the ZnPNPs may exist in various morphological shapes such as nanowires, nanospheres, nanocrystals, nanorectangles, nanotriangles, nanopentagons, nanohexagons, nanoprisms, nanodisks, nanocubes, nanoribbons, nanoblocks, nanotoroids, nanodiscs, nanobarrels, nanogranules, nanowhiskers, nanoflakes, nanofoils, nanopowders, nanoboxes, nanobeads, nanobelts, nano-urchins, nanoflowers, nanostars, tetrapods, their mixtures, and the like. The ZnPNPs are in the form of rectangular flakes. In some embodiments, a surface of the ZnPNPs is in the form of rectangular flakes.

The ZnPNPs include 30-35 wt. %, preferably 31-34 wt. %, and preferably 32-33 wt. % oxygen, 12-18 wt. %, preferably 13-17 wt. %, and preferably 14-16 wt. % phosphorous, and 50-55 wt. %, preferably 51-54 wt. %, and preferably 52-53 wt. % zinc, based on the total weight of the ZnPNPs. In some embodiments, the ZnPNPs include 33-34 wt. %, preferably 33.1-33.9 wt. %, preferably 33.2-33.8 wt. %, preferably 33.3-33.7 wt. %, and preferably 33.4-33.6 wt. % oxygen, 14-15 wt. %, preferably 14.1-14.9 wt. %, preferably 14.2-14.8 wt. %, preferably 14.3-14.7 wt. %, and preferably 14.4-14.6 wt. % phosphorous, and 52-53 wt. %, preferably 52.1-52.9 wt. %, preferably 52.2-52.8 wt. %, preferably 52.3-52.7 wt. %, and preferably 52.4-52.6 wt. % zinc, based on a total weight of the ZnPNPs. In a preferred embodiment, the ZnPNPs include about 33.44 wt. % oxygen, about 14.44 wt. % phosphorus, and about 52.12 wt. % zinc, based on the total weight of the ZnPNPs.

In some embodiments, the ZnPNPs have a polydispersity index (PDI) of 0.8-0.9, preferably 0.81-0.89, preferably 0.82-0.88, preferably 0.83-0.87, and preferably 0.84-0.86. In a preferred embodiment, the ZnPNPs have a polydispersity index of about 0.848.

In some embodiments, the ZnPNPs have a zeta potential of −40 to −30 mV, preferably −39 to −31 mV, preferably −38 to −32 mV, preferably −37 to −33 mV, and preferably 36 to 34 mV. In a preferred embodiment, the ZnPNPs have a zeta potential of about-33.3 mV.

The sample includes RBCs contacted with the organic solvent including DMSO and the ZnPNPs have a reduction in hemolysis 4 to 5 times, preferably 4.1 to 4.9 times, preferably 4.2 to 4.8 times, preferably 4.3 to 4.7 times, and preferably 4.4 to 4.6 times that of a sample including RBCs contacted with an organic solvent including DMSO and not including the ZnPNPs. This decrease in hemolysis displays that the ZnPNPs aid in stabilizing the membranes of RBCs, reducing damage and promoting improved cell integrity.

In some embodiments, the ZnPNPs create a barrier on a surface of the RBCs. In an embodiment, the ZnPNPs create the barrier on the surface of the RBCs through intermolecular forces such as dipole-dipole, ion-dipole, Van der Waals forces, a combination thereof, and the like. In some embodiments, the ZnPNPs create a barrier on a surface of the RBCs covering at least 50%, preferably at least 60%, preferably at least 70%, preferably at least 80%, preferably at least 90%, preferably at least 95%, and preferably at least 99% of the total surface area of the RBCs. In some embodiments, the zinc phosphate nanoparticle barrier on the surface of the RBCs does not allow the DMSO to penetrate the RBCs. In some embodiments, the zinc phosphate nanoparticle barrier on the surface of the RBCs allows less than 5 percent by volume (vol. %), preferably less than 4 vol. %, preferably less than 3 vol. %, preferably less than 2 vol. %, preferably less than 1 vol. %, more preferably less than 0.5 vol. %, and yet more preferably less than 0.1 vol. % DMSO to penetrate the RBCs based on a total volume of the DMSO.

In some embodiments, the sample containing RBCs has a free hemoglobin level of 0.05-0.25 grams per liter (g/L), preferably 0.06-0.24 g/L, preferably 0.07-0.23 g/L, preferably 0.08-0.22 g/L, preferably 0.09-0.21 g/L, preferably 0.1-0.2 g/L, preferably 0.11-0.19 g/L, preferably 0.12-0.18 g/L, preferably 0.13-0.17 g/L, preferably 0.14-0.16 g/L, and more preferably about 0.15 g/L after the contacting.

In some embodiments, 75-95%, preferably 76-94%, preferably 77-93%, preferably 78-92%, preferably 79-91%, preferably 80-90%, preferably 81-89%, preferably 82-88%, preferably 83-87%, preferably 84-86%, and more preferably about 85% of the RBCs in the sample are viable after the contacting.

In some embodiments, the method of reducing hemolysis induced by DMSO further includes sonicating the ZnPNPs in a polar solvent to form a solution and contacting the solution with test cells. In some embodiments, a solution may include fluid mixtures, mixed fluid solidy suspensions, blends, compositions, a combination thereof, and the like. In alternate embodiments, other modes of agitation known to those of ordinary skill in the art, for example, via stirring, swirling, mixing, or a combination thereof may be employed to form the solution. In some embodiments, at least 15%, preferably at least 16%, preferably at least 17%, preferably at least 18%, preferably at least 19%, preferably at least 20%, preferably at least 21%, and more preferably at least 22% of the test cells are viable at a ZnPNPs concentration of 1 mg/mL or less. In a preferred embodiment, about 20% of the test cells are viable at a ZnPNPs concentration of 1 mg/mL or less.

In some embodiments, the polar solvent may include, but is not limited to, water, ethanol, IPA, acetone, acetonitrile, DMSO, ethyl acetate, THF, formic acid, acetic acid, DMF, pyridine, N-methyl-2-pyrrolidone (NMP), propylene carbonate, glycerol, butanol, ethylene glycol, propanol, 1,4-dioxane, sulfolane, a combination thereof, and the like. The water may be tap water, distilled water, bi-distilled water, DW, deionized distilled water, reverse osmosis water, and/or some other water. In a preferred embodiment, the water is DW. In a preferred embodiment, the polar solvent includes water, DMSO, and a phosphate-buffered saline (PBS).

In some embodiments, contacting the solution with the test cells is from 5-50 days, preferably 6-49 days, preferably 7-48 days, preferably 8-47 days, preferably 9-46 days, preferably 10-45 days, preferably 11-44 days, preferably 12-43 days, preferably 13-42 days, preferably 14-41 days, preferably 15-40 days, preferably 16-39 days, preferably 17-38 days, preferably 18-37 days, preferably 19-36 days, preferably 20-35 days, preferably 21-34 days, preferably 22-33 days, preferably 23-32 days, preferably 24-31 days, preferably 25-30 days, preferably 26-29 days, and preferably 27-28 days. In a preferred embodiment, the solution is contacted with the test cells for about 10 days. In another preferred embodiment, the solution is contacted with the test cells for about 20 days. In yet another preferred embodiment, the solution is contacted with the test cells for about 40 days.

In some embodiments, the test cells may include, but are not limited to, mouse fibroblast cells, rat hepatocyte cells, human dermal fibroblast cells, bovine endothelial cells, chicken embryonic cells, human lung carcinoma cells, monkey kidney cells, human breast cancer cells, canine prostate cells, human colon cancer cells, mouse skeletal muscle cells, hamster ovary cells, rabbit corneal cells, human pancreatic cancer cells, human melanoma cells, human mesenchymal stem cells, rat neuronal cells, human osteoblast cells, human neural stem cells, human lung fibroblast cells, human adipose stem cells, human prostate cancer cells, monkey skin cells, human liver cells, rabbit kidney cells, canine neural cells, rat pituitary cells, human leukemia cells, human embryonic stem cells, human adipocytes, mouse stem cells, human chondrocytes, a combination thereof, and the like. In a preferred embodiment, the test cells are human embryonic kidney (HEK 293) cells.

In some embodiments, 105-115%, preferably 106-114%, preferably 107-113%, preferably 108-112%, preferably 109-111%, and preferably about 110% of the test cells are viable at a solution concentration of 0.05-0.08 mg/mL and preferably 0.06-0.07 mg/mL. In some embodiments, 110-115%, preferably 111-114%, and preferably 112-113% of the test cells are viable at a solution concentration of 0.1-0.15 mg/mL, preferably 0.11-0.14 mg/mL, and preferably 0.12-0.13 mg/mL. In some embodiments, 100-110%, preferably 101-109%, preferably 102-108%, preferably 103-107%, preferably 104-106%, and preferably about 105% of the test cells are viable at a solution concentration of 0.2-0.3 mg/mL, preferably 0.21-0.29 mg/mL, preferably 0.22-0.28 mg/mL, preferably 0.23-0.27 mg/mL, and preferably 0.24-0.26 mg/mL. In a preferred embodiment, about 109.9% of the test cells are viable at a solution concentration of about 0.0625 mg/mL. In another preferred embodiment, about 112.4% of the test cells are viable at a solution concentration of about 0.125 mg/mL. In yet another preferred embodiment, about 106.9% of the test cells are viable at a solution concentration of about 0.25 mg/mL.

In some embodiments, the ZnPNPs have a median lethal dose (LD₅₀) in rats of 2400-2600 mg/kg, preferably 2410-2590 mg/kg, preferably 2420-2580 mg/kg, preferably 2430-2570 mg/kg, preferably 2440-2560 mg/kg, preferably 2450-2550 mg/kg, preferably 2460-2540 mg/kg, preferably 2470-2530 mg/kg, preferably 2480-2520 mg/kg, and preferably 2490-2510 mg/kg of body weight. In a preferred embodiment, the ZnPNPs have an LD₅₀ in rats of about 2512 mg/kg of body weight.

In some embodiments, the ZnPNPs have an LD₅₀ in mice of 4800-5200 mg/kg, preferably 4810-5190 mg/kg, preferably 4820-5180 mg/kg, preferably 4830-5170 mg/kg, preferably 4840-5160 mg/kg, preferably 4850-5150 mg/kg, preferably 4860-5140 mg/kg, preferably 4870-5130 mg/kg, preferably 4880-5120 mg/kg, preferably 4890-5110 mg/kg, preferably 4900-5100 mg/kg, preferably 4910-5090 mg/kg, preferably 4920-5080 mg/kg, preferably 4930-5070 mg/kg, preferably 4940-5060 mg/kg, preferably 4950-5050 mg/kg, preferably 4960-5040 mg/kg, preferably 4970-5030 mg/kg, preferably 4980-5020 mg/kg, and preferably 4990-5010 mg/kg of body weight. In a preferred embodiment, the ZnPNPs have an LD₅₀ in mice of about 5000 mg/kg of body weight.

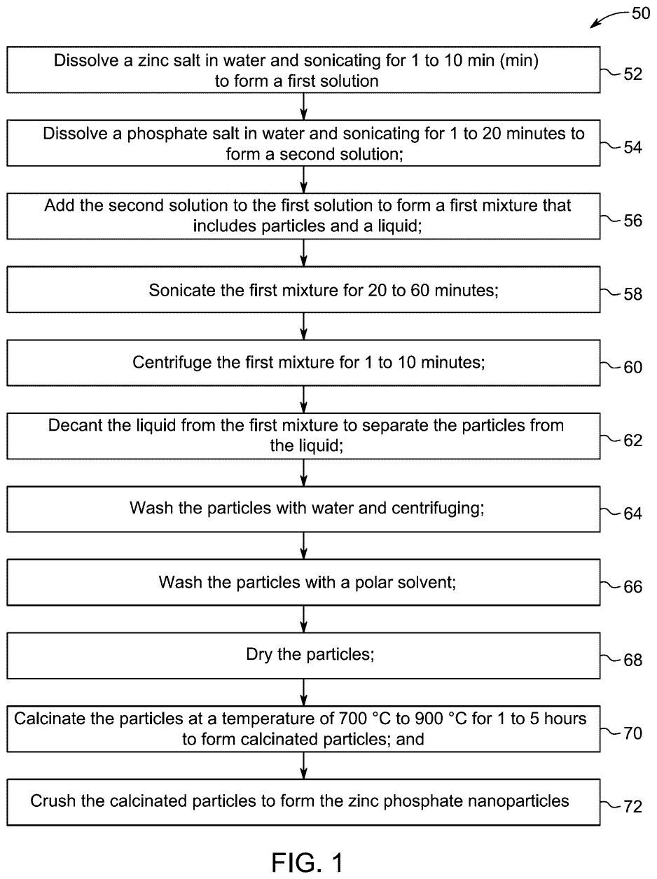

FIG. 1 illustrates a schematic flow chart of a method 50 of forming the ZnPNPs. The order in which the method 50 is described is not intended to be construed as a limitation, and any number of the described method steps can be combined in any order to implement the method 50. Additionally, individual steps may be removed or skipped from the method 50 without departing from the spirit and scope of the present disclosure.

At step 52, the method 50 includes dissolving a zinc salt in water and sonicating for 1 to 10 minutes (min) to form a first solution. In some embodiments, the zinc salt may include, but is not limited to, zinc nitrate, zinc acetate, zinc chloride, zinc sulfate, zinc carbonate, zinc oxide, zinc bromide, zinc iodide, zinc fluorosilicate, zinc tartrate, zinc citrate, zinc gluconate, zinc stearate, zinc picolinate, zinc lactate, zinc benzoate, zinc pyrophosphate, zinc molybdate, zinc borate, zinc chromate, zinc silicate, zinc arsenate, zinc sulfite, zinc ferrocyanide, zinc cyanide, zinc selenite, zinc tellurite, zinc perchlorate, zinc tungstate, zinc oxalate, a combination thereof, and the like. In a preferred embodiment, the zinc salt is zinc nitrate hexahydrate.

In some embodiments, the zinc salt is sonicated in water for 1-10 min, preferably 2-9 min, preferably 3-8 min, preferably 4-7 min, and preferably 5-6 min to form a first solution. In a preferred embodiment, zinc salt is sonicated in water for about 2-3 min. Sonication ensures better dispersion and increased solubility and/or reactivity of the zinc salt in water. The water may be tap water, distilled water, bi-distilled water, deionized water (DW), deionized distilled water, reverse osmosis water, and/or some other water. In a preferred embodiment, the water is DW.

At step 54, the method 50 includes dissolving a phosphate salt in water and sonicating for 1-20 min, preferably 2-19 min, preferably 3-18 min, preferably 4-17 min, preferably 5-16 min, preferably 6-15 min, preferably 7-14 min, preferably 8-13 min, preferably 9-12 min, and preferably 10-11 min to form a second solution.

In some embodiments, the phosphate salt may include, but is not limited to, ammonium phosphate, calcium phosphate, potassium phosphate, sodium phosphate, magnesium phosphate, aluminum phosphate, ferric phosphate, zinc phosphate, sodium tripolyphosphate, disodium phosphate, monosodium phosphate, trisodium phosphate, lithium phosphate, cobalt phosphate, manganese phosphate, barium phosphate, strontium phosphate, lead phosphate, copper phosphate, nickel phosphate, cadmium phosphate, ammonium dihydrogen phosphate, diammonium hydrogen phosphate, dipotassium phosphate, monopotassium phosphate, sodium hexametaphosphate, sodium pyrophosphate, sodium metaphosphate, calcium hydrogen phosphate, aluminum metaphosphate, magnesium hydrogen phosphate, a combination thereof, and the like. In a preferred embodiment, the phosphate salt is sodium phosphate (Na₂HPO₄).

At step 56, the method 50 includes adding the second solution to the first solution to form a first mixture including particles and a liquid. In some embodiments, the second solution may be added to the first solution using various methods such as steady pouring, spraying, stirring during addition, gradual injection, gentle layering, a combination thereof, and the like. In a preferred embodiment, the second solution is introduced to the first solution in a dropwise manner to form a first mixture.

At step 58, the method 50 includes sonicating the first mixture for 20-60 min, preferably 21-59 min, preferably 22-58 min, preferably 23-57 min, preferably 24-56 min, preferably 25-55 min, preferably 26-54 min, preferably 27-53 min, preferably 28-52 min, preferably 29-51 min, preferably 30-50 min, preferably 31-49 min, preferably 32-48 min, preferably 33-47 min, preferably 34-46 min, preferably 35-45 min, preferably 36-44 min, preferably 37-43 min, preferably 38-42 min, more preferably 39-41 min, and yet more preferably about 40 min. In a preferred embodiment, the first mixture is sonicated for 40 min.

At step 60, the method 50 includes centrifuging the first mixture for 1-10 min, preferably 2-9 min, preferably 3-8 min, preferably 4-7 min, preferably 5-6 min, and more preferably about 5 min. In a preferred embodiment, first mixture is centrifuged for 5 min. Centrifugation helps to separate the components of a mixture by spinning it at high revolutions per min and causes heavier particles to settle at the bottom.

At step 62, the method 50 includes decanting the liquid from the first mixture to separate the particles from the liquid. The liquid is drained out, leaving the particles behind, when the mixture has had time to settle.

At step 64, the method 50 includes washing the particles with water and centrifuging. In some embodiments, the particles may be washed with water between 1-10 times, preferably 2-9 times, preferably 3-8 times, preferably 4-7 times, and preferably 5-6 times. In a preferred embodiment, the particles are washed with water about 3 times.

At step 66, the method 50 includes washing the particles with a polar solvent. In some embodiments, the polar solvent may include, but is not limited to, water, ethanol, IPA, acetone, acetonitrile, DMSO, ethyl acetate, THF, formic acid, acetic acid, DMF, pyridine, NMP, propylene carbonate, glycerol, butanol, ethylene glycol, propanol, 1,4-dioxane, sulfolane, a combination thereof, and the like. In a preferred embodiment, the polar solvent is methanol.

At step 68, the method 50 includes drying the particles. The particles may be dried by using heating appliances such as ovens, microwaves, autoclaves, hot plates, heating mantles and tapes, oil baths, salt baths, sand baths, air baths, hot-tube furnaces, hot-air guns, a combination thereof, and the like. In a preferred embodiment, the particles are dried using a hot air oven. In some embodiments, the particles may be dried for 12-36 hours (h), preferably 13-35 h, preferably 14-34 h, preferably 15-33 h, preferably 16-32 h, preferably 17-31 h, preferably 18-30 h, preferably 19-29 h, preferably 20-28 h, preferably 21-27 h, preferably 22-26 h, preferably 23-25 h, and more preferably about 24 h. In a preferred embodiment, the particles are dried for 24 h.

At step 70, the method 50 includes calcinating the particles at a temperature of 700-900° C. for 1 to 5 h to form calcinated particles. Calcination is carried out by heating a material to a high temperature, under a restricted supply of ambient oxygen. This is performed to remove impurities or volatile substances and to incur thermal decomposition. Calcination may be carried out in a furnace preferably equipped with a temperature control system, which may provide a heating rate of up to 50° C./min, preferably up to 40° C./min, preferably up to 30° C./min, preferably up to 20° C./min, preferably up to 10° C./min, and preferably up to 5° C./min. In some embodiments, calcination may occur at a temperature of 700-900° C., preferably 710-890° C., preferably 720-880° C., preferably 730-870° C., preferably 740-860° C., preferably 750-850° C., preferably 760-840° C., preferably 770-830° C., preferably 780-820° C., preferably 790-810° C., and more preferably about 800° C. for 1-5 h, preferably 2-4 h, and more preferably about 3 h. In a preferred embodiment, calcination of the first mixture occurs at a temperature of 800° C. for 3 h to form calcinated particles.

At step 72, the method 50 includes crushing the calcinated particles to form the ZnPNPs. Exemplary techniques used for crushing calcinated particles to form the ZnPNPs include, but are not limited to, manual methods (e.g., mortar), ball mill, hammer mill, vibrating grinder, planetary mill, ultrasonic crusher, jet mill, disc mill, roller mill, cryogenic grinder, bead mill, a combination thereof, and the like. In a preferred embodiment, crushing the calcinated particles to form the ZnPNPs is done with mortar and pestle.

### EXAMPLES

The following examples describe and demonstrate a method of reducing hemolysis induced by dimethyl sulfoxide (DMSO) in red blood cells (RBCs) using zinc phosphate nanoparticles (ZnPNPs). The examples are provided solely for illustration and are not to be construed as limitations of the present disclosure, as many variations thereof are possible without departing from the spirit and scope of the present disclosure.

### Example 1: Materials and Methods

Chemical reagents used in the present disclosure were of analytical grade and purchased from Sigma Aldrich (St. Louis, MO, USA). ZnPNPs were synthesized using zinc nitrate hexahydrate (Zn(NO₃)₂·6H₂O), sodium phosphate (Na₂HPO₄), methanol, and deionized water (DW) as precursors.

### Example 2: Synthesis of ZnPNPs

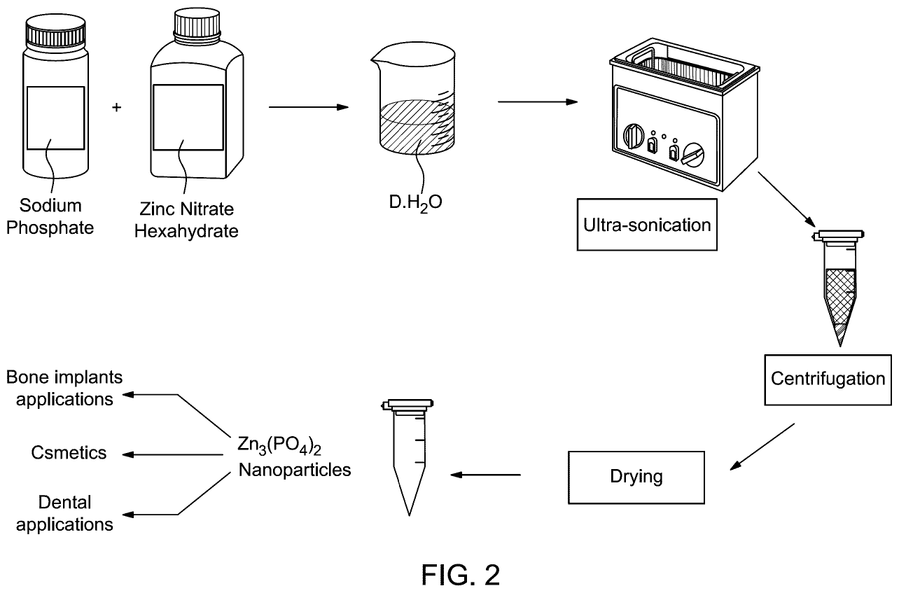

Zn(NO₃)₂·6H₂O and Na₂HPO₄ were used to synthesize the ZnPNPs with DW serving as a solvent. The sono-chemical method, which applied ultrasound waves to dissolve, degas, and facilitate interactions among material components, ensured no toxic products or gases were generated. For synthesizing the ZnPNPs, a batch preparation was performed. Approximately 0.5 grams (g) of Zn(NO₃)₂·6H₂O was weighed and dissolved in 30 milliliters (mL) of DW, labelled as solution A, and prepared in two beakers. The solution was sonicated for 2 to 3 minutes (min). Separately, 0.3 g of Na₂HPO₄ (dibasic anhydrous) was dissolved in 10 mL of DW, labeled as solution B, and prepared in two beakers. The solution was sonicated until the salt completely dissolved. Further, solution B was added dropwise to solution A, preceding to the immediate formation of a white solution. The suspension was covered with foil and sonicated for 40 min. The resulting mixture was transferred to 10 mL Falcon tubes, ensuring no residues were left behind, and centrifuged for 5 min. The sediment particles were washed three times with water to remove any undissolved materials, followed by centrifugation. The particles were then washed twice with methanol and dried in an oven at temperature 80 degrees Celsius (C) for 24 hours (h). Furthermore, the dried sample was then collected and calcined at 800° C. for 3 h. The calcined product was crushed using a mortar and pestle, collected, and transferred into an Eppendorf tube (microtube) and labelled as ZnPNPs. FIG. 2 is a schematic diagram depicting synthesis steps of the ZnPNPs.

### Example 3: Characterization of NPs

ZnPNPs were characterized using various analytical instruments, including scanning electron microscopy (SEM), energy-dispersive X-ray spectroscopy (EDX), X-ray diffraction (XRD), transmission electron microscopy (TEM), and zeta potential analysis. These techniques were employed to assess the crystallinity, purity, morphology, and elemental composition of the ZnPNPs. X-ray diffraction (Rigaku, Japan) was utilized to examine the phases of ZnPNPs within the range of 20 degrees (°) to 70° at a scanning speed of 0.9° per minute. SEM and EDX analyses provided detailed information on the elemental composition and surface characteristics of the ZnPNPs. TEM analysis was conducted to evaluate the morphology and particle size of the ZnPNPs. The zeta size and zeta potential of the ZnPNPs were measured using dynamic light scattering (DLS) with a Malvern Zetasizer instrument (Malvern, United Kingdom). Prior to analysis, the samples were thoroughly dispersed in a saline solution through ultrasonication to ensure uniformity.

### Example 4: Surface Morphology and Characterization Results

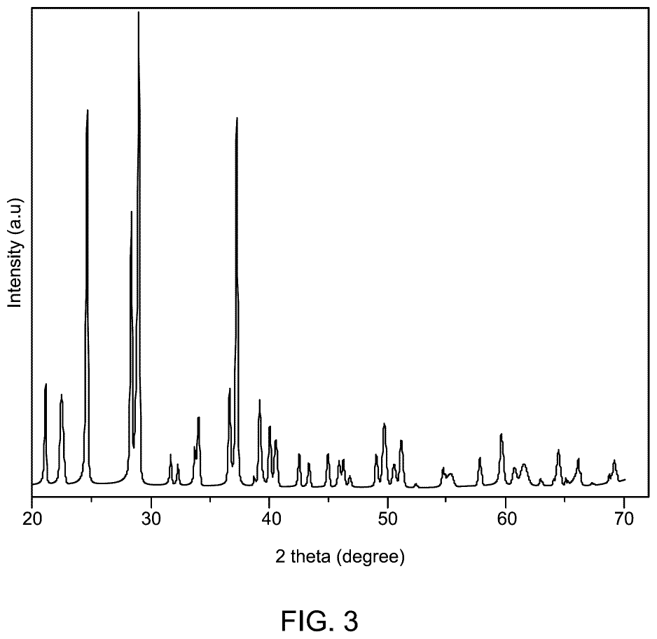

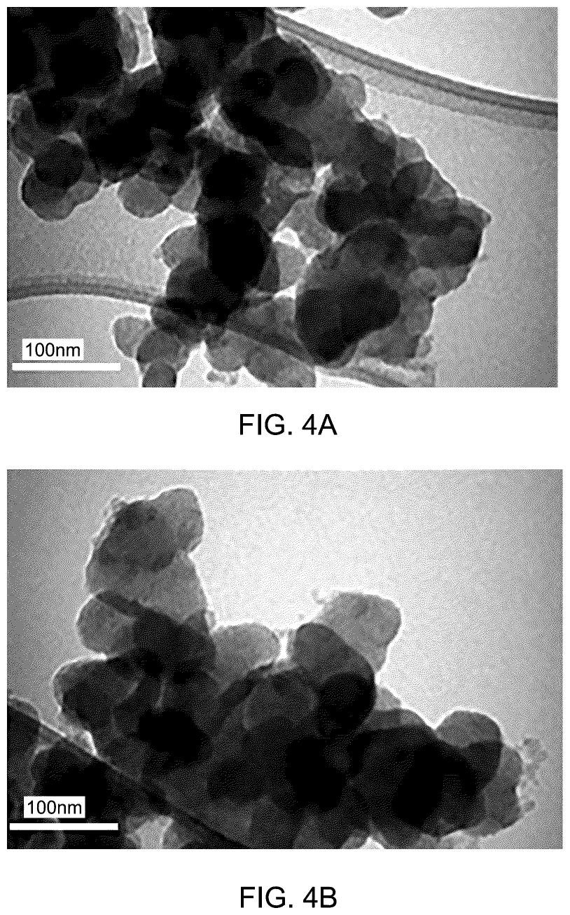

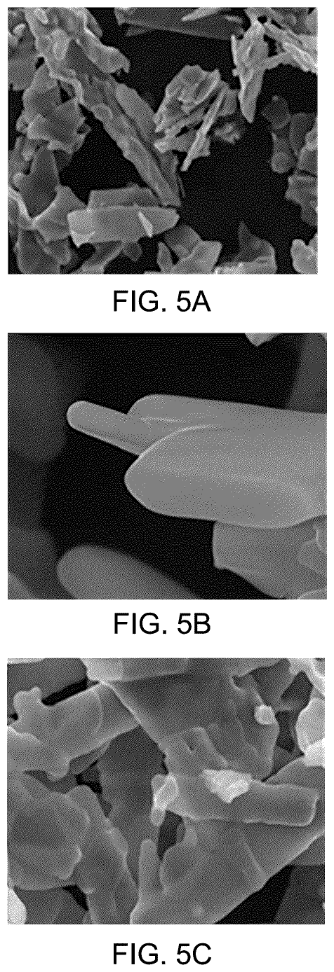

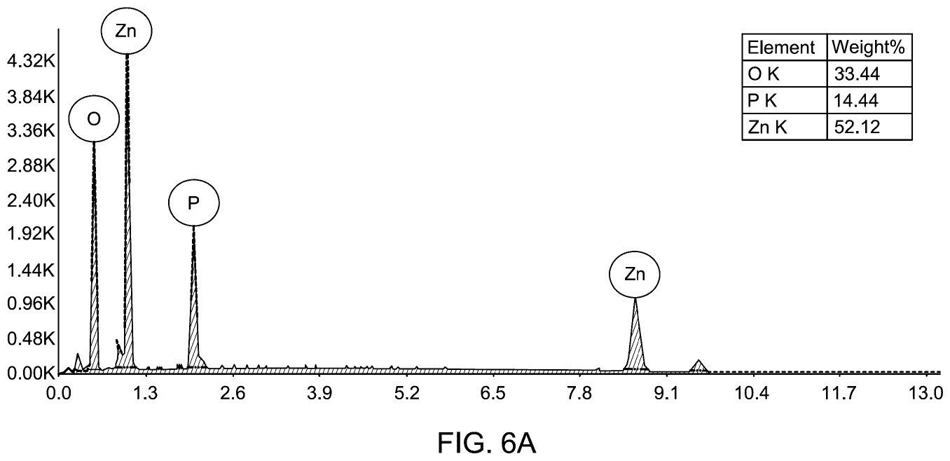

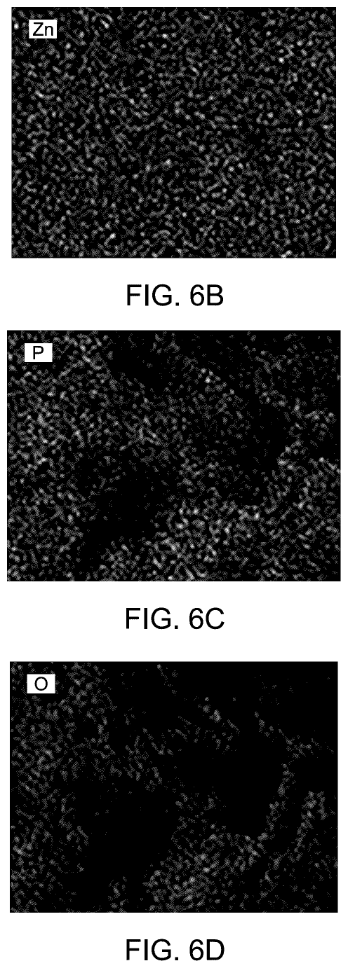

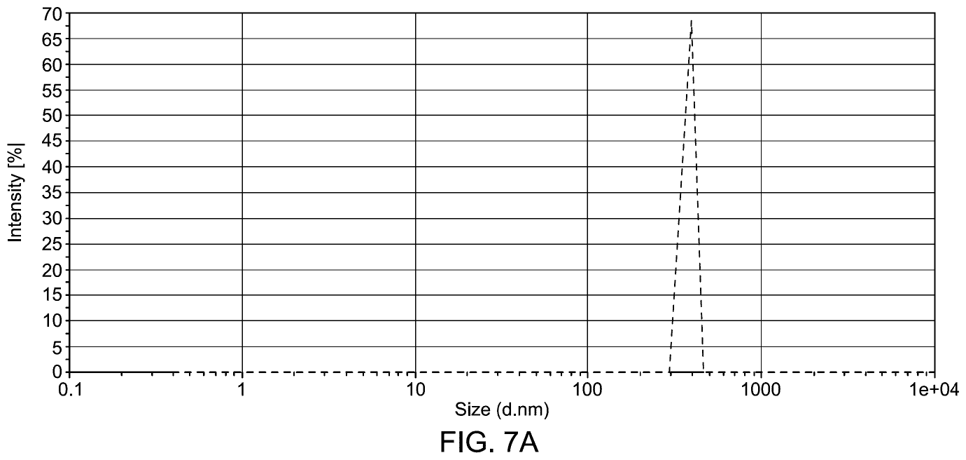

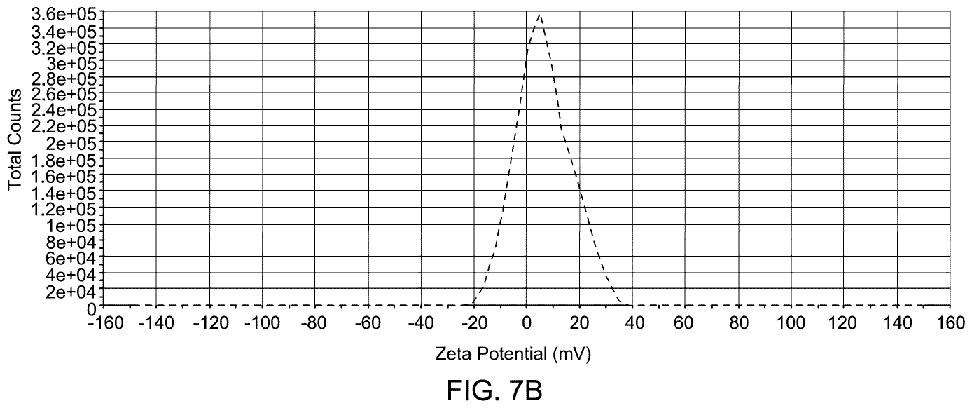

The XRD pattern of the synthesized ZnPNPs, as shown in FIG. 3, correlates well with ICDD card no. 04-013-3009 and revealed the crystalline nature of the material. TEM analysis was conducted to evaluate the morphology and size of the ZnPNPs, revealing an average particle size of approximately 43 nanometers (nm), as shown in FIG. 4A. The TEM images depicted a variety of shapes and surface morphologies, including asymmetrical shapes, semi-spherical particles, and heterojunctions, as shown in FIGS. 4A-4B. SEM images demonstrated that the surface of the ZnPNPs resembles fine flakes, as shown in FIGS. 5A-5C. No further roughness or bumps were observed on the surfaces of the ZnPNPs. EDX measurements were performed at multiple locations to assess the elemental distribution of zinc (Zn), phosphorous (P), and oxygen (O). The EDX spectra and elemental mapping, as shown in FIG. 6A, confirmed the chemical composition of ZnPNPs. The weight percentages (wt. %) of O, P, and Zn were observed. The corresponding elemental mapping images are shown in FIG. 6B, FIG. 6C, and FIG. 6D, showing distribution of Zn, P, and O, respectively. Zeta size and zeta potential analyses were carried out to determine the surface charge and colloidal stability of the synthesized ZnPNPs, as shown in FIGS. 7A-7B. The average hydrodynamic diameter of the ZnPNPs was measured as 1652 nm, with a polydispersity index of 0.848. The zeta potential was recorded as −33.3 millivolts (mV), indicating moderate stability, as shown in FIG. 7B. FIG. 7A is a plot showing particle size distribution of ZnPNPs.

The XRD pattern results of ZnPNPs confirmed the effective synthesis of highly crystalline ZnPNPs, as shown in FIG. 3. These findings align with previous synthesis and characterizations of ZnPNPs [Sadeghi-Aghbash, M. and Rahimnejad, M, ZnP NPs: A review on physical, chemical, and biological synthesis and their applications, Curr Pharm Biotechnol, 2022, 23, 10, 1228-1244, which is incorporated herein by reference in its entirety]. Prior evaluations involved the synthesis of ZnPNPs using different precursors, including hydrogen phosphate, diphosphate, and triphosphate, highlight variations in preparation methods and outcomes [Horky, P. et al., ZnP-based nanoparticles as a novel antibacterial agent: In vivo study on rats after dietary exposure, Journal of Animal Science and Biotechnology, 2019, 10, 17, which is incorporated herein by reference in its entirety]. The first two NPs exhibited spherical shapes with smaller diameters of 477 nm and 521 nm, respectively, compared to the zinc phosphate nanoparticle size reported in the present disclosure. Previous research observed diameters of 452 nm and 1035 nm for two NPs, which may be attributed to the formulation based on anions being larger than cations. This resulted in irregular shapes and the formation of small aggregates, a feature not observed in the present disclosure. The polydispersity indices of the NPs ranged from 0.16 to 0.19.

The size of the ZnPNPs was around 43 nm, which was examined using TEM, as shown in FIG. 4A-4B. The particles displayed an asymmetrical, semi-spherical shape and showed heterojunctions between particles. FIG. 5A shows a SEM image of the synthesized ZnPNPs. FIGS. 5B-5C shows SEM image highlighting a specific area with a flake-like structure of the synthesized ZnPNPs. The morphology of ZnPNPs, examined by SEM, resembled fine peels or chips, as shown in FIGS. 5A-5C. EDX measurements were conducted to evaluate the distribution of the three atoms in the ZnPNPs including zinc (Zn), phosphate (P), and oxygen (O), and corresponding peaks were generated. Additionally, the values of O, P, and Zn, as determined by SEM-EDX analysis, are shown in FIGS. 6A-6D. The results provided high-quality chemical properties and small size and shape with no deformities, protrusions, or bumps on the surface of the ZnPNPs. The determined sizes and properties indicate that ZnPNPs are candidates for biocompatibility, as they have mild effects on blood circulation and capillaries. Additionally, they may be internalized into cells without causing damage to animal or human cells. The size of NPs plays a role in biocompatibility [Preedia Babu, E. et al., Size-dependent uptake and hemolytic effect of zinc oxide nanoparticles on erythrocytes and biomedical potential of ZnO-ferulic acid conjugates, Scientific Report, 2017, 7, 4203, which is incorporated herein by reference in its entirety]. The particle size obtained from the dynamic light scattering (DLS) analysis was larger than that observed in the SEM and TEM analysis. The differences observed may be explained by the fact that DLS measures the hydrodynamic size of particles, including the diffuse layer surrounding them, and the size obtained from SEM/TEM analysis was the size of the ZnPNPs. The polydispersity index was 0.848, indicating a heterogeneous particle size distribution. To evaluate the stability of ZnPNPs in suspension, zeta potential was examined. The zeta potential of the ZnPNPs was recorded at −33.3 millivolts (mV). NPs tend to agglomerate and are considered unstable when the zeta potential is between −10 mV and +10 mV. Conversely, NPs are considered stable when the zeta potential was greater than ±30 mV [Clogston, J. D., & Patri, A. K., Zeta potential measurement, Methods Mol Biol, 2011, 697, 63-70, which is incorporated herein by reference in its entirety].

The present disclosure describes on how nanostructures interact with biological systems, to determine how chemical and physical characteristics of nanostructures, such as shape, surface, size, composition, and aggregation, relates to the induction of toxic biological responses, as determined through in vivo evaluation. Examining the interaction of nanoparticles (NPs) in biological systems has become an issue, particularly for mitigating risks to human health, considering rapid and extensive advancements in nanomedicine. A range of factors influence cellular uptake of NPs; for example, when NPs pass through a biological fluid, such as blood, their surface is immediately coated with a layer of biomolecules, including proteins and lipids.

The ZnPNPs were studied in vitro using a human embryonic kidney cell line (HEK293). A hemolysis experiment was also performed on rat blood samples. Subsequently, these materials were tested on mice and rats. Observations were made on these treated animals and a median lethal dose (LD₅₀) was determined.

The effect of NPs absorption into various types of cells is exclusive to materials ranging from 50 to 200 nm in size. The absorption was facilitated by surface modifications, such as positively or negatively charged side groups on amino acids, peptides, or polymers. Understanding parameters that influence cellular uptake, including size, surface characteristics, cell type, medium, cell division, and endocytic routes, allows for selection of NPs and cells for in vitro and in vivo applications [Salatin, S. et al., Effect of the surface modification, size, and shape on cellular uptake of nanoparticles, Cell Biology International, 2015, 39, 8, 881-890, which is incorporated herein by reference in its entirety].

To assess the toxicity of the ZnPNPs, the solubility of the particles was tested by dissolving 3 milligrams (mg) in 1 milliliter (mL) of DW, dimethyl sulfoxide (DMSO), and phosphate-buffered saline (PBS). ZnPNPs are inorganic compounds and did not dissolve immediately in the three solvents; therefore, a sonicator was used to dissolve the particles in a DMSO solution at four different concentrations, including 5 mg/mL, 3 mg/mL, 1 mg/mL, and 0.25 mg/mL, for 40 min. The solutions were examined in a normal cell line, specifically human embryonic kidney (HEK293) cells, using an 3-[4,5-dimethylthiazolyl-2]-2,5-dimethyltetrazolium bromide (MTT) assay.

Cell-based assays were used to evaluate chemicals that affect cell proliferation or directly induce cytotoxicity, leading to cell death. MTT assays measure the results of cell growth and assess the harmful effects of the materials. Various techniques may be used to determine the quantity of viable eukaryotic cells. In the present disclosure, the MTT assay was used. This assay relies on the action of mitochondrial reductase to convert the water-soluble yellow dye MTT into insoluble purple formazan. The intensity of the formazan indicates the presence of live cells. The concentration of cells was measured using a plate reader spectrophotometer at an optical density (OD) of 570 nm [Kumar P. et al., Analysis of cell viability by the MTT assay, Cold Spring Harbor Protocols, 2018, 6, which is incorporated herein by reference in its entirety]. The HEK293 cell line was subcultured in a 60 millimeter (mm) subculture petri dish and incubated overnight at temperature 37° C. in a 5% CO₂ incubator.

The cytotoxicity of the ZnPNPs was determined using the human embryonic kidney (HEK293) cell line, which was obtained from the American type culture collection (ATCC) in Manassas, Virginia, United States.

Revived and sub-cultured cells in the medium of a 60 mm petri dish were removed by suction. The cells were washed with 2 mL of PBS and aspirated. Then, 500 microliters (μL) of trypsin enzyme was added to detach the cells from the bottom of the plate. The petri dish was incubated for 3 to 5 min at 37° C. in a 5% CO₂ incubator. Following incubation, the trypsin was neutralized by adding 5 mL of the medium to prevent detaching the cells. The cell suspension was transferred into a 15 mL tube and centrifuged for 5 min at 1300 revolution per minute (rpm) and 20° C., the supernatant was then removed. Further, 1 mL of fresh medium was added to the cell pellet and thoroughly re-suspended. A mixture of 10 μL from the suspension and 10 μL of trypan blue dye was placed in a microtube and mixed. Furthermore, 10 μL of this mixture was added to a slide chamber, which was inserted into the cell-counting instrument. Live and dead cells for the HEK293 cell line were then measured, as listed in Table 1.

TABLE 1
Data collected from the cell counting instrument, including
total concentration and percentages of live and dead cells

| EK293 | Data collected |
|---|---|
| Total concentration | 6.98 × 10⁶ 1/mL |
| Live cells (%) | 95% − 6.64 × 10⁶ 1/mL |
| Dead cells (%) | 5% − 3.40 × 10⁶ 1/mL |

Calculation for cell seeding in a 96-well plate was performed using the volume of the cell suspension in μL with the following formula:

$$\\ Volume of cell suspension in µL = \frac{number of cells wanted}{number of cells present} \times 1000 = \frac{20 , 000}{6 , 640 , 000} \times 1000 = 3.0124819 µL \\ 3.0124819 µL \times 80 wells = 240.9638552 µL ( total volume of cell suspension ) \\ 100 \times 80 wells = 8000 µL ( media ) \\ 8000 µL of media - 240.9638552 µL is a total volume of cell suspension = 7 , 759.4 µL of total volume of media is used$$

241 μL of a cell suspension was added to 7,759.4 μL of media and 100 μL was distributed in each well. The 96-well plate was then incubated for 24 h in a 5% CO₂ incubator. Each well contained 15,000 cells.

After seeding the cells, they were treated with the nanomaterial (NM), i.e., the ZnPNPs, and 5-fluorouracil, a chemotherapeutic drug used to kill cancer cells. The latter was used as a reference drug for comparing toxicity with ZnPNPs.

After 24 h of incubation, a stock solution of the ZnPNPs was prepared by dissolving 5 mg of the sample in 1 mL of a 50% mixture of dimethyl sulfoxide (DMSO) and Dulbecco's modified Eagle medium (DMEM). Five different dilutions of the stock solution and five control concentrations were then prepared, as listed in Table 2.

For the reference drug, four concentrations of 5-Fluorouracil, including 5 mg, 3 mg, 1 mg, and 0.5 mg, were prepared by dissolving them in 1 mL of DMSO. Media was removed from the wells, and 100 μL of each concentration of controls, ZnPNPs, and 5-Fluorouracil were added to each well. The 96-well plate was read by a plate reader spectrophotometer at 570 nm and incubated at 37° C. for 24 h.

TABLE 2
Different concentrations of NPs and controls with quantities of solvents

| Number of | | |
|---|---|---|
| dilutions | Concentration (mg/mL) | Control (%) |
| First | 1 | 10 |
| | 200 μL of Stock + 800 μL of DMEM | 100 μL of DMSO + 900 μL DMEM |
| Second | 0.5 | 5 |
| | 100 μL of Stock + 900 μL of DMEM | 50 μL of DMSO + 950 μL DMEM |
| Third | 0.25 | 2.5 |
| | 50 μL of Stock + 950 μL of DMEM | 25 μL of DMSO + 975 μL DMEM |
| Fourth | 0.125 | 1.25 |
| | 25 μL of Stock + 975 μL of DMEM | 12.5 μL of DMSO + 987.5 μL |
| | | DMEM |
| Fifth | 0.0625 | 0.625 |
| | 12.5 μL of Stock + 987.5 μL of DMEM | 6.75 μL of DMSO + 993.75 μL |
| | | DMEM |

One day later, the ZnPNPs, control, and chemo-drug solutions were removed, and the cells were washed with 100 μL of PBS. A resuspension in 100 μL of PBS was performed, and OD readings were taken before adding the MTT dye.

Subsequently, 10 μL of MTT and 90 μL of Dulbecco's modified eagle medium (DMEM) was applied to the cells and incubated (protected from light) for a duration of 4 h. After incubation, the media was removed, and 100 μL of solubilization solution DMSO was added to dissolve the formazan crystals formed due to mitochondrial reduction. Optical density (OD) absorbance readings were measured using a plate reader spectrophotometer at a wavelength (λ) of 570 nm.

The calculation of the viability percentage was performed by using the formula:

$$\\ \frac{number of treated cells}{number of control cells} \times 100$$

At a concentration of 1 mg/mL of ZnPNPs:

$$\\ \frac{0.215}{0.986} \times 100 = 21.8 \%$$

(viable cells)

$$\\ At 0.5 mg / mL : \frac{0.328}{0.063} \times 100 = 19.2 \% \\ At 0.25 mg / mL : \frac{2.808}{1.463} \times 100 = 106.9 \% \\ At 0.125 mg / mL : \frac{3.492}{3.106} \times 100 = 112.4 \% \\ At 0.0625 mg / mL : \frac{1.387}{1.262} \times 100 = 109.9 \%$$

To assess the bio-application of the ZnPNPs, particularly in biomedical fields, it is beneficial to understand both their toxicity and biocompatibility. The hemolysis test is one assays used to observe the effects of NPs or other drugs on RBCs. Hemolysis involves destruction of RBC membranes, resulting in hemoglobin leakage and the release of cellular content, which may lead to several complicated health issues [Alfareed T. M. et al., Biocompatibility and colorectal anti-cancer activity study of nano-sized BaTiO₃ coated spinel ferrites, Scientific Reports, 2022, 12, 1, 1-18, which is incorporated herein by reference in its entirety]. The free hemoglobin produced from the disrupted RBCs was collected from the supernatant and measured using a spectrophotometer; however, the degree of hemolysis may also be measured using a plate reader [Sæbø, I. P. et al., Optimisation of the hemolysis assay for the assessment of cytotoxicity, International Journal of Molecular Sciences, 2023, 24, 3, 2914, which is incorporated herein by reference in its entirety].

A rat blood sample was collected, purified, and incubated with the ZnPNPs to evaluate whether the ZnPNPs induces membrane damage in RBCs. Initially, approximately 10 mL of blood was collected from a rat and drawn from the abdominal vena cava into an ethylenediaminetetraacetic acid (EDTA) tube to prevent coagulation. The sample was then centrifuged at 1,500 rpm for 10 min, after which the supernatant was removed without disturbing the RBCs. After centrifugation, the RBCs were washed with PBS and centrifuged for an additional 10 min. The washing and centrifuging steps were repeated twice. The washed erythrocytes were then re-suspended in PBS.

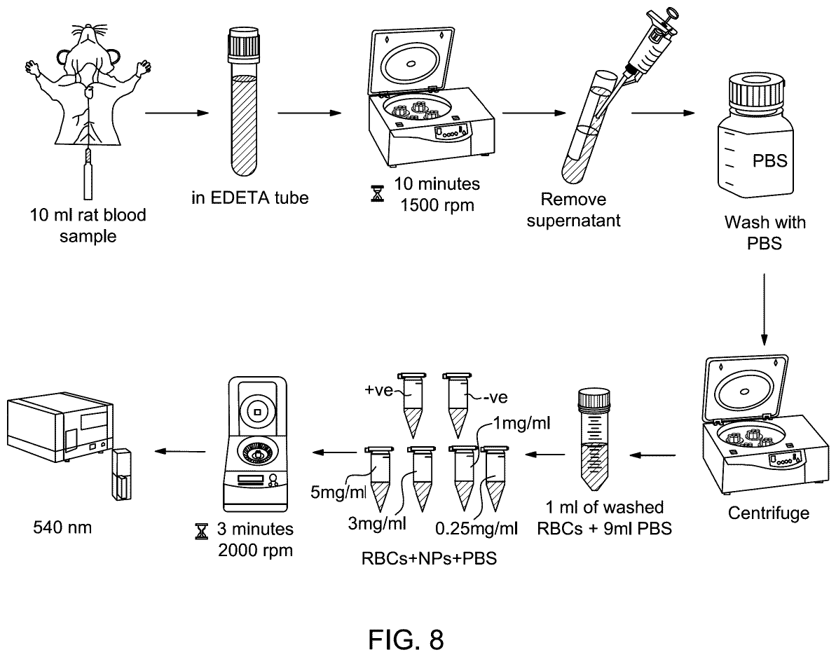

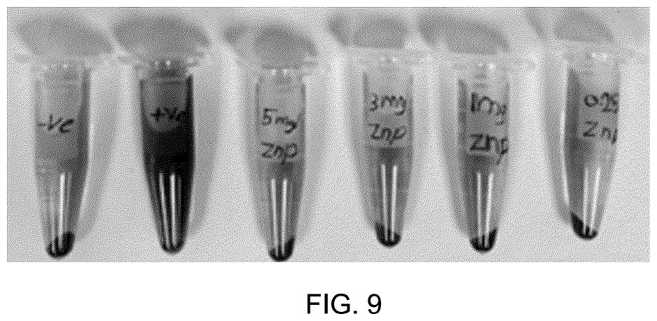

1 mL of the washed RBCs was transferred into a 15 mL Falcon tube and mixed with 9 mL of PBS. Next, in four micro-tubes, 1 mL of PBS, 500 μL of RBCs, and 5 μL of ZnPNPs were gently mixed. The ZnPNPs were dissolved in normal saline at four concentrations, including 5 milligrams per milliliter (mg/mL), 3 mg/mL, 1 mg/mL, and 0.25 mg/mL. For the positive control, which was completely hemolyzed, 50 mg of sodium dodecyl sulfate (SDS) was dissolved in 1 mL of PBS solution, and 5 μL of this mixture was added to 500 μL of RBCs and 1 mL of PBS. In the negative control, only SDS was replaced with PBS, but the quantities of RBCs and PBS remained equal to those in the positive control. All micro-tubes were incubated for 20 min at 37° C. and then centrifuged at 2,000 rpm for 3 min. FIG. 8 is a schematic illustration of a hemolysis test protocol using microtubes containing rat RBCs treated with ZnPNPs. The supernatant (hemoglobin) was collected in different tubes. FIG. 9 depicts microtubes containing rat RBCs treated with ZnPNPs. The samples were transferred to cuvettes for absorbance readings at 540 nm using a UV-visible spectrophotometer. The cuvettes were washed with PBS between each sample. Hemolysis percentage calculations were performed using Equation 1.

$$\\ \begin{matrix}\frac{OD negative - OD sample}{OD positive - OD negative} & ( Eq . 1 )\end{matrix}$$

To obtain an LD₅₀ of ZnPNPs, an examination was carried out in compliance with the Organization for Economic Cooperation and Development (OECD) test guideline TG 425, 27 for the testing of chemicals [Organization for Economic Co-operation Development (OECD), Guidance Document on Acute Oral Toxicity Testing 420, Organization for Economic Co-operation Development, 2008; and Organisation for Economic Co-operation Development, Test no. 425: Acute oral toxicity: up-and-down procedure, Organisation for Economic Co-operation Development, 2008, which are incorporated herein by references in their entireties]. Male Sprague-Dawley rats, aged 6 to 8 weeks and weighing 200-220 g, were used. The experimental protocol was approved by the Ethical Committee for Animal Experimentation of Imam Abdulrahman Bin Faisal University. Animals were randomly housed in groups in cages with stainless steel covers to allow acclimatization. The environmental conditions were 12 h day/night cycle, temperature 22±2° C., relative humidity 60±10%, standard diet and water ad libitum. A pilot study according to OECD TG 425 (Acute oral toxicity) was performed to determine the starting dose. Rats were exposed to the ZnPNPs by a single oral gavage. A single animal was dosed in sequence. The first animal receives a dose one step below an estimate of the LD₅₀. If the animal survives, the second animal receives a higher dose. If the first animal dies, the second animal receives a lower dose. Based on this, the LD₅₀ was determined using a conventional method [Ahmed, M. et al., Acute toxicity (lethal dose 50 calculation) of herbal drug somina in rats and mice, Pharmacology & Pharmacy, 2015, 6, 185-189, which is incorporated herein by reference in its entirety]. A total of 80 healthy Sprague-Dawley male rats weighing 200 g-220 g, were randomly assigned to 8 experimental groups (n=10). Based on the pilot study, the doses utilized were saline (vehicle control), 20 mg/kg, 40 mg/kg, 80 mg/kg, 160 mg/kg, 300 mg/kg, 1000 mg/kg, and 2000 mg/kg of each material in a fixed volume of 0.5 mL. The animals were observed 6 h, 12 h, 24 h, and 48 h after treatment, and the number of dead animals was noted. A Hodge and Sterner scale was used, as listed in Table 3, to evaluate toxicity [Hodge, A and Sterner, B., Toxicity classes, Canadian Centre for Occupational Health and Safety, 2005, which is incorporated herein by reference in its entirety]. The percentage of mortality was plotted against the log concentration of different doses. A log concentration that causes 50% mortality is determined from the curve. This value was transferred into an antilog to give the value of LD₅₀ of the substance.

TABLE 3
Hodge and sterner toxicity scale

| No. | Term | LD₅₀ (Rat, Oral) | |
|---|---|---|---|
| 1 | Extremely Toxic | Less than 1 mg/kg | |
| 2 | Highly Toxic | 1-50 | mg/kg |
| 3 | Moderately Toxic | 50-500 | mg/kg |
| 4 | Slightly Toxic | 500-5000 | mg/kg |
| 5 | Practically Non-Toxic | 5000-15000 | mg/kg |

White albino mice, including male and female animals, weighing between 20 g and 25 g, were used in a separate experiment. They were housed in large, airy cages, with 10 animals per cage, allowing unrestricted access to food and water. An approximate LD₅₀ was initially determined through a pilot study, employing the “staircase method,” which involved two animals per dose and incrementally increasing doses of ZnPNPs. Subsequently, five doses were selected for determining the oral LD₅₀ and administered to five groups of albino mice, each group containing 10 animals. One group of mice including 6 animals were given 0.5 mL and 1.0 mL of saline orally as controls. The animals were observed for the first 2 h, followed by observations at the 6th and 24th hour for any signs of toxicity. After 24 h, the number of deceased mice in each group was recorded. The percentage of animals that had died at each dose level was transformed to log and then LD₅₀ was determined.

Irregular treatment-related measurements indicate variations in toxicological and/or pharmacological consequences. These changes may occur in tissue morphology, as determined by histological analysis, or in a variety of in vivo measurements, such as clinical chemistry analysis that is used for identifying effects of therapy or any chemicals.

An acute oral toxicity analysis was performed according to the Organization of Economic Co-Operation and Development (OECD) guideline 420 for testing of chemicals to determine if the ZnPNPs are toxic [Organization for Economic Co-operation Development (OECD), Guidance Document on Acute Oral Toxicity Testing 420, Organization for Economic Co-operation Development, 2008; and Organisation for Economic Co-operation Development, Test no. 425: Acute oral toxicity: up-and-down procedure, Organisation for Economic Co-operation Development, 2008, which are incorporated herein by references in their entireties]. Male and female rats, aged 6 weeks to 8 weeks and fasted for 16 h, were used. The ZnPNPs were administered only once orally at a single dose of 300 mg/kg and 2000 mg/kg at a rate of 20 mL/kg to both the sexes of rats, which included a total number of 10 rats (n=10) including five males and five females. The control group only received the vehicle. All rats were then allowed free access to food and water and were observed for 24 h, with special care given to the first 4 h and once daily for 14 days for any signs of acute toxicity. Visual observations were conducted once daily for 14 days. On the 15th day, all animals were anesthetized by an intraperitoneal injection of ketamine. Blood samples were collected by cardiac puncture into non-heparinized tubes for biochemical analysis and kept at room temperature for a minimum of 90 min and then centrifuged, at approximately 3000 rpm, for 10 min, at room temperature to obtain serum. Clinical chemistry analysis was conducted with a Toshiba 200 FR NEO chemistry analyzer (Toshiba Co., Japan). Blood serum from ten exposed rats and eight control rats included in the biological effect examination was analyzed. Sera from two control rats were excluded from the analysis due to hemolysis. The concentrations of 10 metabolites, including albumin, chloride, cholesterol, creatinine, phosphate, potassium, sodium, blood urea nitrogen, uric acid and total protein, and the activities of three enzymes, alanine aminotransferase (ALT), alkaline phosphatase (ALP) and aspartate aminotransferase (AST), were measured with standard protocols for blood sample screenings (Roche Diagnostics). Liver function was evaluated based on the serum levels of alkaline phosphatase, alanine aminotransferase and aspartate aminotransferase. Nephrotoxicity was determined by measuring the concentrations of blood urea nitrogen and uric acid in the serum.

Visual observations of mortality, changes in physical appearance, behaviors such as salivation and lethargy, injuries or illnesses, sensory function tests including tail pinch, approach and touch response, pupillary reflex, acoustic startle response, grip strength, and motor activity were conducted once daily for 14 days.

The results were expressed as a mean from all samples±standard deviation. Statistical significance was determined by examining the basic differences among groups using ANOVA and Duncan's multiple-range test (DMRT) for all parameters. The differences with P<0.05 were considered as significant.

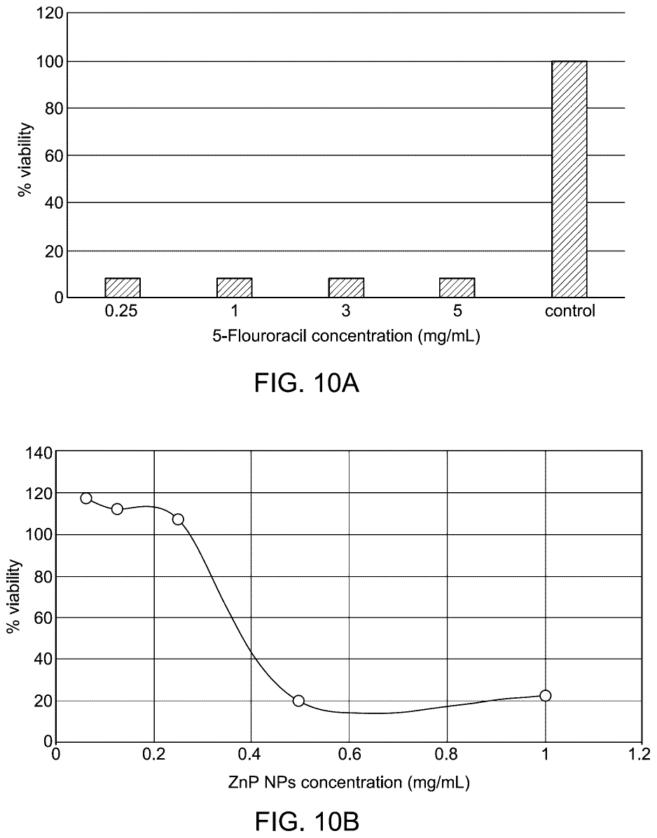

ZnPNPs exhibited a non-toxic effect on HEK293 cells at concentrations below 1 mg/mL. Lower concentrations of the ZnPNPs resulted in higher cell viability. In comparison to the reference chemotherapeutic drug 5-fluorouracil, which was evaluated for its efficacy in cell destruction, the chemical drug caused over 90% cell toxicity, with fewer than 8% live cells observed. The data demonstrated the high toxicity of the chemical drug on normal cells (HEK293). In contrast, cells treated with ZnPNPs showed over 100% proliferation at lower concentrations of 0.25 mg/mL, 0.125 mg/mL, and 0.0625 mg/mL, while approximately 20% proliferation was observed at 1 mg/mL. Cell toxicity was directly proportional to the concentration of the ZnPNPs, with higher concentrations resulting in increased toxicity, as shown in FIGS. 10A-10B. FIG. 10B depicts the cell viability (%) treated with ZnPNPs.

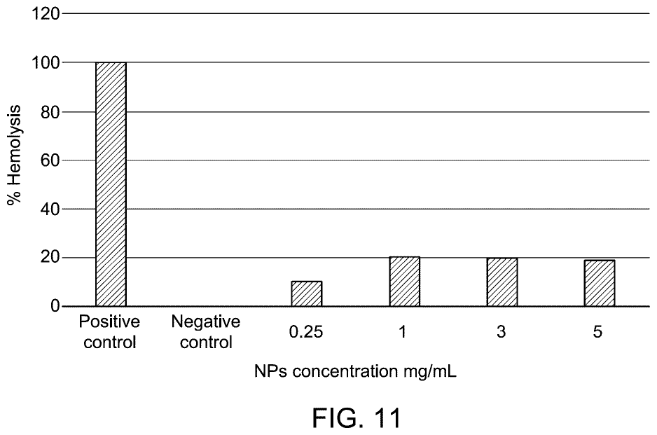

A hemolysis test was conducted to evaluate the destruction of erythrocytes, revealing that the ZnPNPs caused approximately 20% hemolysis at concentrations of 1 mg/mL, 3 mg/mL, and 5 mg/mL. FIG. 11 depicts a graph of hemolysis percentage (%) of the ZnPNPs. These concentrations were identified as high at the ZnPNPs level, as shown in FIG. 11. Values of LD₅₀ (In vivo) of ZnPNPs and the acute toxicity values of various concentrations of ZnPNPs are listed in Table 4.

TABLE 4
Values of LD₅₀ of different concentrations of ZnPNPs in mice and rats

| | Concentration (mg/kg) | | | | | | | | | | |
|---|---|---|---|---|---|---|---|---|---|---|---|
| | 100 | 100 | 400 | 400 | 1000 | 1000 | 1500 | 1500 | 2500 | 2500 | |
| Mice and Rat | Ex | De | Ex | De | Ex | De | Ex | De | Ex | De | LD₅₀ |
| Mice | 10 | 1 | 10 | 2 | 10 | 4 | — | — | — | — | |
| Rats | 10 | 0 | 10 | 0 | 10 | 0 | 10 | 1 | 10 | 5 | |
| Conc. mg/kg | 2500 | 2500 | 3000 | 3000 | 4000 | 4000 | 5000 | 5000 | 8000 | 8000 | |
| Mice | — | — | — | — | — | — | 10 | 5 | 10 | 6 | 5000 |
| Rats | 10 | 5 | 10 | 7 | 10 | 9 | — | — | — | — | 2512 |

Ex: Exposed mice or rats,
De: Dead mice or rats

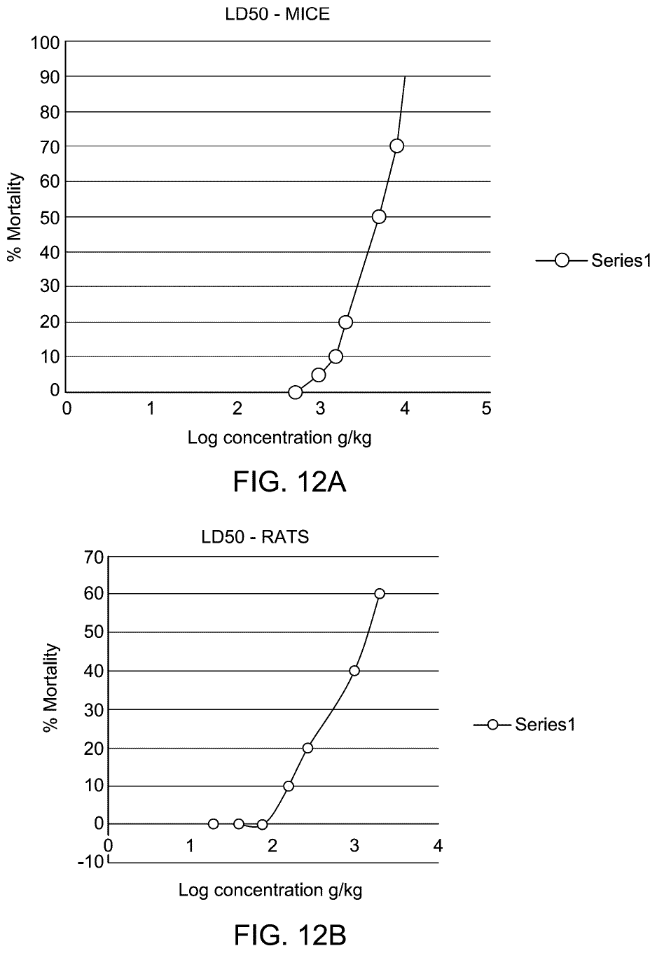

According to the Hodge and Sterner toxicity scale, the obtained LD₅₀ value for the ZnPNPs was 5000 mg/kg body weight in mice and 2512 mg/kg in rats, categorizing them as slightly toxic, as shown in FIG. 12A and FIG. 12B, respectively.

In functional observations, including sensory function tests (tail pinch, approach and touch response, pupillary reflex, and acoustic startle response), grip strength, and motor activity, no treatment-related changes were observed during the study. Intoxicated animals frequently exhibit abdominal inflation and are unable to walk normally, displaying slow, writhing, and exaggerated movements of the neck and tail, which progress to gasping and eventual death.

The clinical blood chemistry analyses showed no significant differences in serum metabolites or enzyme activities between the control group and the exposed rats in the biological effect study, as listed in Table 5. Enzyme activities and concentration of blood urea nitrogen and uric acid were significantly (P<0.05) increased in rats dosed with 2000 mg/kg body weight. Table 6 shows serum concentrations of ACTH and cortisone hormones (adrenal gland functions) in mice after treatment with ZnPNPs for different durations. Table 7 shows serum concentrations of protein and liver enzymes in mice after treatment with ZnPNPs for different durations.

TABLE 5
Mean values (±SD) of clinical chemistry parameters
of rat blood serum dosed orally with ZnPNPs

| Metabolite/Enzyme | Control | | 300 mg/kg | | 2000 mg/kg | |
|---|---|---|---|---|---|---|
| Albumin (g/L) | 10.9 | (0.4) | 11.7 | (0.3) | 9.9 | (0.4) |
| Chloride (mmol/L) | 95.9 | (1.4) | 94.8 | (1.5) | 93.3 | (1.6) |
| Cholesterol (mmol/L) | 3.2 | (0.4) | 3.3 | (0.2) | 3.2 | (0.3) |
| Creatinine (mmol/L) | 16.6 | (1.6) | 17.5 | (1.5) | 16.8 | (1.8) |
| Phosphate (mmol/L) | 5.2 | (0.2) | 5.3 | (0.3) | 5.5 | (0.4) |
| Potassium (mmol/L) | 7.3 | (1.2) | 8.1 | (1.2) | 8.3 | (1.7) |
| Sodium (mmol/L) | 140.5 | (11.7) | 141.5 | (9.3) | 141.5 | (8.3) |
| Total protein (g/L) | 64.7 | (2.3) | 60.4 | (1.1) | 60.4 | (3.1) |
| Uric acid (mmol/L) | 344.4 | (71.9) | 380.6 | (76) | 522.4 | (54.9) |
| Blood urea nitrogen (mmol/L) | 5.2 | (0.8) | 4.8 | (0.6) | 8.2 | (0.7)* |
| Alkaline phosphatase, ALP (U/L) | 110.3 | (25.0) | 94.5 | (34.7) | 125.5 | (21.5)* |
| Alanine aminotransferase, ALT (U/L) | 83.54 | (21.6) | 84.14 | (25.3) | 100.7 | (31.5)* |
| Aspartate aminotransferase, AST (U/L) | 146.5 | (33.7) | 150.2 | (31.9) | 196.3 | (32.3)* |

*P < 0.05

TABLE 6
Serum concentrations of ACTH and cortisone hormones
(adrenal gland functions) in mice after treatment
with ZnPNPs for different durations

| | ACTH (pg/mL) | Cortisol (ng/mL) |
|---|---|---|
| Group | Mean ± SD | Mean ± SD |
| Control | 8.1 ± 0.1 | 82.9 ± 0.4 |
| Treated for 10 days (w/ZnPNPs) | 11.1 ± 0.3* | 135 ± 1.3* |
| Treated for 20 days (w/ZnPNPs) | 8.3 ± 0.1 | 86.1 ± 0.6 |
| Treated for 40 days (w/ZnPNPs) | 8.3 ± 0.1 | 83.4 ± 0.4 |

The results were analyzed using an ANOVA test, with asterisks (*) indicating significant differences compared to the control group. In the control group, the mean cortisol level was lower than the mean ACTH level; however, in the group treated with ZnPNPs for 10 days, the mean cortisol level showed a significant increase compared to the control group, rising from 82.9±0.4 nanograms per milliliter (ng/mL) to 135±1.3 ng/mL, along with an increase in the mean ACTH level. In contrast, the groups treated for 20 days and 40 days exhibited mean cortisol levels comparable to the control group, while the mean ACTH levels remained relatively consistent across treatment durations. Observations indicate that treatment with ZnPNPs for 10 days led to a significant increase in cortisol levels, indicating a stimulatory effect on cortisol production, while the mean ACTH levels increased in parallel. Longer treatment durations did not result in sustained elevation of cortisol levels.

TABLE 7
Serum concentrations of protein and liver enzymes in mice
after treatment with ZnPNPs for different durations

| | | | | Total protein |
|---|---|---|---|---|
| Group (mean) | AST (U/dL) | ALT (U/dL) | ALP (U/dL) | (g/dL) |
| Control | 89.14 ± 9.3 | 35.57 ± 4.3 | 854.29 ± 69.3 | 5.228 ± 1.332 |
| Treated for 10 days | 90.5 ± 8.8 | 35.55 ± 5.3 | 850 ± 79.3 | 5.233 ± 1.200 |
| (w/ZnPNPs) | | | | |
| Treated for 20 days | 92.43 ± 9.6 | 35.14 ± 6.4 | 855.71 ± 89.2 | 5.228 ± 1.119 |
| (w/ZnPNPs) | | | | |
| Treated for 40 days | 94.14 ± 8.9 | 36 ± 6.3 | 844.29 ± 91.3 | 5.214 ± 1.301 |
| (w/ZnPNPs) | | | | |

A significant difference was observed in the mean concentrations of aspartate aminotransferase (AST) and alanine aminotransferase (ALT) among the groups (p<0.05), while the mean alkaline phosphatase (ALP) levels showed no significant differences (p>0.05). Additionally, the mean concentrations of total protein exhibited significant variation among the groups (p<0.05). These observations indicate that the treatment duration may influence the levels 10 of AST, ALT, and total protein in mice, while ALP levels remain relatively stable.

TABLE 8
Serum concentrations of renal enzymes in mice after
treatment with ZnPNPs for different durations

| Group | Creatinine (mg/dL) | Urea (mg/dL) | Uric Acid (mg/dL) |
|---|---|---|---|
| Control | 0.22 ± 0.02 | 54.8 ± 8.78 | 6.07 ± 0.23 |
| Mean ± SD | | | |
| Treated for 10 days | 0.28 ± 0.05* | 50.38 ± 6.41* | 6.05 ± 0.26 |
| Mean ± SD | | | |
| Treated for 20 days | 0.27 ± 0.05 | 51.68 ± 6.74 | 5.97 ± 0.42 |
| Mean ± SD | | | |
| Treated for 20 days | 0.25 ± 0.05 | 49.88 ± 4.72 | 6.04 ± 0.15 |
| Mean ± SD | | | |

Further, the analysis of serum concentrations of renal enzymes in mice subjected to treatment with ZnPNPs for various durations demonstrated significant alterations in creatinine and urea levels relative to the control group. Table 8 shows serum concentrations of renal enzymes in mice after treatment with ZnPNPs for different durations. Specifically, a marked increase in creatinine levels and a significant decrease in urea levels were observed following 10 days of treatment. Uric acid concentrations remained consistent across all treatment groups. These findings indicate the impact of ZnPNPs on renal function, particularly in the modulation of creatinine and urea metabolism. The results contribute to the understanding of physiological responses associated with nanoparticle administration.

TABLE 9
Serum concentrations of thyroid hormones in mice after
treatment with ZnPNPs for different durations

| Group | T3 (ng/mL) | T4 (μg/mL) | TSH (μgU/mL) |
|---|---|---|---|
| Control | 0.85 ± 0.03 | 4.77 ± 0.26 | 0.22 ± 0.02 |
| Mean ± SD | | | |
| Treated for 10 days | 0.82 ± 0.04 | 4.66 ± 0.11* | 0.22 ± 0.02 |
| Mean ± SD | | | |
| Treated for 20 days | 0.83 ± 0.03 | 4.78 ± 0.08 | 0.20 ± 0.03 |
| Mean ± SD | | | |
| Treated for 40 days | 0.82 ± 0.03 | 4.67 ± 0.08 | 0.20 ± 0.05 |
| Mean ± SD | | | |

Mean serum concentrations of T3, T4, and TSH were analyzed in mice treated with ZnPNPs for 10 days, 20 days, and 40 days, along with a control group. Table 9 shows serum concentrations of thyroid hormones in mice after treatment with ZnPNPs for different durations. No missing samples were reported in the treated groups, except for one missing sample in the group treated for 10 days. ANOVA tests were conducted to evaluate differences in mean hormone levels across the treatment durations. Significant differences, indicated by asterisks (*), were observed compared to the control group. The results indicate effects of ZnPNPs treatment on thyroid hormone levels in mice, particularly after 10 days of exposure.

No significant differences were observed in T3 levels between the control group and the groups treated with ZnPNPs for 10 days, 20 days, or 40 days; however, a significant decrease in T4 levels was observed in mice treated with ZnPNPs for 10 days compared to the control group. No significant differences were detected in T4 levels between the control group and the groups treated with ZnPNPs for 20 days or 40 days. In terms of TSH levels, no significant differences were observed between the control group and any of the treatment groups. The decrease in T4 levels after 10 days of ZnPNPs treatment indicated an impact on thyroid function, although the lack of significant differences in T3 and TSH levels indicate that the effects of ZnPNPs on thyroid function may have been limited or transient. While the results indicate an influence of ZnPNPs on thyroid function, evidenced by the decrease in T4 levels after 10 days of treatment, further studies to evaluate long-term implications of ZnPNPs exposure on thyroid health may be performed.

ZnPNPs particles are well-tolerated biochemical compounds, as they naturally occur in the body, particularly in bones and teeth, where they contribute to the formation of hydroxyapatite. In the present disclosure, a nano-formulation was prepared to enhance their physiological and therapeutic effects as a nanomedicine.

The in vitro non-toxic effect of ZnPNPs on the viability of normal cells (HEK293) has been determined, demonstrating its safety compared with a highly toxic effect (90%) of the reference drug, 5-fluorouracil—a chemotherapeutic drug. FIG. 10A is a bar graph of cell viability (%) treated with 5-fluorouracil drug.

Preclinical toxicological examinations are needed for using any molecule/compound for pharmacological and therapeutic reasons [Sayyad, M. et al., Acute toxicity profiling of the ethyl acetate fraction of Swietenia macrophylla seeds and in-vitro neuroprotection studies, Saudi Pharm. J., 2017, 25, 2, 196-205, which is incorporated herein by reference in its entirety]. Toxicological evaluations are also needed to establish drug dosage determination at preclinical drug development stages [Mohs, R. C. and Greig, N. H., Drug discovery and development: role of basic biological research, Alzheimers Dement (NY), 2017, 3, 4, 651-657, which is incorporated herein by reference in its entirety].

Acute in vivo toxicity is involved in determination of the LD₅₀ that represented the dose which is lethal to 50% of the experimentally tested animals [Raj, J. et al., Determination of median lethal dose of combination of endosulfan and cypermethrin in wistar rat, Toxicology International, 2013, 20, 1, 1-5, which is incorporated herein by reference in its entirety]. The present disclosure determined the lethal dose of ZnPNPs in male Wister rats and mice by oral route.

In the present disclosure, values of LD₅₀ acute toxicity of ZnPNPs were determined in accordance with OECD TG 401. According to the Hodge and Sterner toxicity scale, the obtained value of LD₅₀ of ZnPNPs was 5000 mg/kg body weight of mice and 2512 mg/kg of rats, which was of the slightly toxic category.

New Zealand white rabbits (N=3) were treated with 100 mg of ZnPNPs instilled into a conjunctival sac of the left eye in accordance with OECD TG 405. The other eye served as untreated control. The eyes (unrinsed) were examined at 1 h, 24 h, 48 h, and 72 h, post-application. Non-irritating results were observed in 2 animals, while slight irritation of conjunctiva and chemosis was noted within 48 h post-application. No iris or corneal lesions or conjunctival discharge observed (European Chemicals Agency, 2017).

Other zinc salts were use to evaluate toxicity of zinc oxide nanoparticles (ZnONPs) in exposed mice with diameters of ZnO being 20 nm and 120 nm. ZnO (20 nm) demonstrated that the LD₅₀ was greater than 5000 mg/kg body weight. ZnO (120 nm) administered to rats demonstrated an LD₅₀ value greater than 2000 mg/kg body weight to less than 5000 mg/kg body weight [Wang, B. et al., Acute toxicological impact of nano- and submicro-scaled zinc oxide powder on healthy adult mice, Journal of Nanoparticle Research, 2008, 10, 263-276, which is incorporated herein by reference in its entirety]. Zinc nitrate administered to rats and mice showed that the effective LD₅₀ values were 133 mg/kg body weight and 110 mg/kg body weight, respectively. Zinc sulfate and zinc acetate treated rats and mice showed effective LD₅₀ values of 200 mg/kg body weight and 316 mg/kg body weight and 162 mg/kg body weight and 108 mg/kg body weight, respectively [Domingo, J. L. et al., Acute zinc intoxication: comparison of the antidotal efficacy of several chelating agents, Veterinary and human toxicology, 1988, 30, 3, 224-228, which is incorporated herein by reference in its entirety].

An objective of the present disclosure is to collect the results on different systems of animals to assess risks of the chemicals at high and ambient exposure levels. Traditionally, high-dose exposure was the main objective of toxicity investigations to determine LD₅₀ levels. But in a new era, focus transformed into molecular levels to determine the mechanism of action and target identification of the toxicity. The U.S. NRC in 2007 introduced a new vision and strategy in toxicity studies to evaluate toxicity pathways. These pathways are normal cellular pathways but can be perturbed by exposure to chemicals. Toxicity pathway indicators provide information regarding the risk of a substance to human health [Bhattacharya, S. et al., Toxicity testing in the 21 century: defining new risk assessment approaches based on perturbation of intracellular toxicity pathways, PLoS one, 2011, 6, 6, e20887, which is incorporated herein by reference in its entirety].

An acute oral toxicity study at 2000 mg/kg was conducted to test if a material is non-toxic [Prabu, P. C. et al., Acute and sub-acute oral toxicity assessment of the hydroalcoholic extract of Withania somnifera roots in wistar rats, Phytother Res., 2013, 27, 8, 1169-1178, which is incorporated herein by reference in its entirety]. The acute oral toxicity study at 2000 mg/kg dose, also called the limit test, is used to test the toxicity of the compound. ZnPNPs at 2000 mg/kg dose did not show any effect, indicating that it is non-toxic. The complete survival and general state of health of the rats together with analyses of blood serum metabolites and enzyme activities, indicated that ZnPNPs was not acutely toxic to rats. Clinical blood chemistry analyses showed no significant differences in serum metabolites or enzyme activities between the control group and rats exposed to 300 mg/kg and 2000 mg/kg. At the 2000 mg/kg dose level there was an increased activity of alanine aminotransferase, alkaline phosphatase and aspartate aminotransferase indicating liver damage. Blood urea nitrogen and uric acid concentrations in the serum were also increased, indicating renal damage.

Rats were fed with hydrated ZnPNPs in a dose of 2000 mg/kg body weight among other zinc salts NP treatments. The antioxidant status of rat kidney, liver, and blood was determined after zinc NPs treatments. Overall, zinc formulations had no effect on the antioxidant state of rats. On the reverse side, ZnPNPs are capable of producing ROS, resulting in oxidative stress [Horky, P. et al., ZnP-based nanoparticles as a novel antibacterial agent: In vivo study on rats after dietary exposure, Journal of Animal Science and Biotechnology, 2019, 10, 17, which is incorporated herein by reference in its entirety].

Histological examination of tissues from rats treated with zinc NPs revealed no difference in liver damage between treated and untreated control animals.

In the present disclosure, 20% of RBCs showed hemolysis when incubated with different concentrations of the ZnPNPs: 1 mg/mL, 3 mg/mL, and 5 mg/mL. This is an unacceptable result for safety issues. This may be attributed to the penetration of ZnPNPs into mostly weak or old RBCs, increasing their osmotic pressure, which leads to cell lysis, explosion, or dissolution of membranous phospholipids by the ZnPNPs. While 80% of the cells showed resistance, this finding agrees with silver NPs inducing hemolysis at concentrations of more than 700 μg/mL (0.7 mg/mL) and causing a 50% hemolytic effect [Choi, J. et al., Physicochemical characterisation and in vitro hemolysis evaluation of silver nanoparticles, Toxicological Sciences, 2011, 123, 1, 133-143, which is incorporated herein by reference in its entirety]. According to the ASTM E2524-08 standard, a percent hemolysis greater than 5% indicates that the test material causes RBC damage, this condition was met for the silver NPs at a particle concentration of 70 μg/mL. This indicated that the high dose used should be decreased tenfold to reach the levels used in in vitro studies. Testing RBCs is a method for examining the toxicity of NPs, as RBCs have no nucleus and are fragile cells in the body.

Three types of Zn-NPs were examined and were found to have effects on human health due to the imbalance of homeostasis of trace elements and ions in tissues [Park, E. J. et al., Comparison of distribution and toxicity of different types of zinc-based nanoparticles, Environ Toxicol., 2017, 32, 4, 1363-1374, which is incorporated herein by reference in its entirety]. Additionally, the surface coating of ZnO NPs with phosphate and sulfide may not decrease toxicity due to the higher participation level of ZnO NPs in the intestines, at least in part.

The size-dependent interactions of ZnO NPs on RBCs and their effect on cell viability, reactive oxygen species (ROS) production, DNA damage, and morphological alteration has been studied [Preedia Babu, E. et al., Size-dependent uptake and hemolytic effect of zinc oxide nanoparticles on erythrocytes and biomedical potential of ZnO-ferulic acid conjugates, Scientific Reports, 2017, 7, 4203, which is incorporated herein by reference in its entirety]. Size, stability, charge, and water solubility were determined using zeta potential and other methods. ZnO NPs showed a size-dependent effect on RBCs and hemoglobin, especially with sizes less than 50 nm.

Upon the addition of ZnCl₂ to artificial saliva, NPs with a mean radius of 1.1 nm±0.2 nm and a size distribution width of 0.7 nm±0.2 nm were observed, and they formed aggregates with radii of gyration of 37±1 nm [Saloga, P. E. J. et al., ZnP NPs Produced in Saliva, Eur. J. Inorg. Chem., 2020, 3654-3661, which is incorporated herein by reference in its entirety]. De novo ZnPNPs may also form in vivo if food and cosmetics products containing the permitted zinc salt concentrations are ingested, which could be a source of unintentional mineral formation; therefore, ZnPNPs may be naturally present in the body.

The present disclosure aims to comprehensively examine the synthesized ZnPNPs both in vitro and in vivo. An objective was to assess their toxicological impact through the synthesis of ZnPNPs and analysis of their chemical characteristics, structure, and morphology using techniques such as XRD, SEM, TEM, and DLS/zeta potential. The ZnPNPs exhibited a non-toxic effect on HEK293 cells when the concentration of the ZnPNPs was below 1 mg/mL. A hemolysis test was performed to investigate the destruction of erythrocytes, revealing that the ZnPNPs caused approximately 20% hemolysis at concentrations of 1 mg/mL, 3 mg/mL, and 5 mg/mL, indicating a very low hemolytic effect. Additionally, the LD₅₀ value of ZnPNPs in rats were determined to be 2512 mg/kg body weight, classifying it as slightly toxic. In the present disclosure, compositions of hydrated ZnPNPs demonstrates no harmful physiological effects on vital organs, including the liver, kidneys, thyroid gland, and adrenal glands.

In the present disclosure, a formulation of phosphate-based zinc NPs (ZnPNPs) was synthesized and tested. The formulation of zinc NPs based on phosphates with irregular morphology was prepared. TEM was used to examine the morphology and size of Zn₃(PO₄)₂. The average size was about 43 nm, as calculated from the TEM image. TEM also showed that the nanoparticle was asymmetrical in shape, semispherical, and included heterojunctions in between. SEM demonstrated the surface of the ZnPNPs appeared as fine flakes. The ZnPNPs had in vitro 20% hemolysis of rat RBCs at a concentration of 1 mg/mL. The MTT assay (cell viability) was performed on the normal cell line of human embryonic kidney (HEK293). The ZnPNPs showed a non-toxic effect on the HEK293 cells when the concentration of ZnPNPs was lower than 1 mg/mL. The concentration of the ZnPNPs is directly proportional to cell toxicity with a higher concentration leading to a higher toxicity. The highest in vitro inhibitory effect of the ZnPNPs was observed against the viability of cells. After the successful in vitro testing, the in vivo LD₅₀ was determined in rats and mice. Values of LD₅₀ acute toxicity of the ZnPNPs were determined. According to the Hodge and Sterner toxicity scale, the obtained value of LD₅₀ of ZnPNPs in rats was 2512 mg/kg body weight, which fell under the slightly toxic category. In mice, the LD₅₀ was 5000 mg/kg body weight, which was non-toxic. The clinical blood chemistry analyses showed no statically significant differences in serum metabolites or enzyme activities between the control group and the exposed rats dosed with 300 mg/kg body weight; however, enzyme activities and concentrations of blood urea nitrogen and uric acid were increased in rats dosed with 2000 mg/kg body weight. The results indicated that ZnPNPs were nontoxic at a dose of 1 mg/mL in vitro and at a dose of 300 mg/kg body weight in vivo in rats.

An objective of the present disclosure was to evaluate the toxicity of ZnPNPs in comparison to DMSO and determine whether the ZnPNPs suspended in DMSO have any protective effect on RBCs, particularly in preventing hemolytic effects caused by DMSO.

DMSO, at concentrations typically used in biological systems, may induce hemolysis in RBCs, increasing osmotic fragility and free hemoglobin levels [Cadwallader, D. E. and Drinkard, J. P., Behavior of Erythrocytes in Various Solvent Systems IV: Water Dimethylsulfoxide, Journal of Pharmaceutical Sciences, 1967, 56, 5, 583-586; and Yi, X., et al., Toxic effects of dimethyl sulfoxide on red blood cells, platelets, and vascular endothelial cells in vitro, FEBS Open Bio, 2017, 7, 4, 485-494, which are incorporated herein by references in their entireties]. At a 0.6% DMSO concentration, hemolysis significantly increased after 6 h of exposure, with free hemoglobin levels rising to 0.64 g/L±0.19 g/L, compared to the control group, which showed levels of 0.09 g/L±0.05 g/L. This comparison indicated the toxic effect of DMSO on RBCs, highlighting the need to investigate whether ZnPNPs could mitigate these effects.

In contrast to DMSO alone, the present disclosure demonstrated that ZnPNPs suspended in DMSO reduced the hemolytic effect. The experimental setup involved incubating RBCs with ZnPNPs in 0.6% DMSO for the same 6-h period. Hemolysis was assessed by measuring free hemoglobin in the supernatant after centrifugation.

Results showed that the presence of ZnPNPs lowered the free hemoglobin levels compared to the DMSO-only treatment, indicating that the ZnPNPs had a protective effect on the RBC membrane. Specifically, the ZnPNPs+DMSO-treated samples exhibited free hemoglobin levels of 0.15±0.07 g/L, which is lower than the 0.6% DMSO-treated group (0.64 g/L±0.19 g/L).

The protective effect of ZnPNPs may be attributed to their ability to stabilize the RBC membrane, preventing the osmotic imbalance and membrane disruption typically caused by DMSO. Additionally, the surface properties of ZnPNPs could interact with the RBC membrane, creating a barrier that reduces DMSO's ability to penetrate and cause hemolysis.

Further examinations were certified to explore the exact mechanism by which ZnPNPs protect RBCs and to assess whether this protective effect was consistent across different concentrations and exposure times.

TABLE 10
Comparison between this study and the three listed studies,
focusing on the effects of NPs on RBCs hemolysis

| Feature | 3* | 1* | 2* | 4* | Present study |
|---|---|---|---|---|---|
| NPs Type | Zinc oxide | Silver | Argovit ™ | Xyloglucan- | ZnPNPs |
| | nanoparticles | nanoparticles | Silver | block- | |
| | (ZnONPs) | (AgNPs) with | nanoparticles | polycaprolactone | |
| | | polyvinyl | (AgNPs) | (XGO-PCL) | |
| | | pyrrolidone | | copolymer NPs | |
| | | and citrate | | | |
| | | coatings | | | |
| NPs Size | <50 nm, 50-100 | 28.08 nm, as | Not | 150 nm, | About 43 nm, as |
| | nm, >100 nm | determined by | explicitly | specifically for | indicated by |
| | | (TEM) | mentioned | drug delivery | TEM analysis |
| Hemolytic | Hemolysis | AgNPs induced | Bell-shaped | Hemolytic | 20% hemolysis |
| Effect | dependent | hemolysis at all | hemolysis | ratio less than 1%, | observed at |
| | on size and | concentrations | curve; | indicating | higher |
| | concentration; <50 | tested (10, 20, | higher | good blood | concentrations |
| | nm ZnONPs | 40 μg/mL) | hemolysis | compatibility | (1, 3, and 5 |
| | had the | | for diabetic | | mg/mL), |
| | highest | | erythrocytes; | | suggesting low |
| | hemolytic | | increased | | hemolytic effect |
| | activity | | hemolysis | | |
| | | | at lower pH | | |
| | | | levels (5.6) | | |
| Dose | Maximum | AgNPs induced | Sharp | No significant | No significant |
| Causing | hemolysis at | significant | hemolysis | hemolysis, | hemolysis at 5 |
| Max. | 800 μg/mL | hemolysis at all | increase at | even at high | mg/mL |
| Hemolysis | for <50 nm | tested | higher | nanoparticle | (significantly |
| | ZnONPs | concentrations | concentrations; | concentrations | higher doses |
| | | (10, 20, 40 | bell- | | compared to |
| | | μg/mL) | shaped | | other studies) |
| | | | curve | | |
| | | | indicates | | |
| | | | unusual | | |
| | | | hemolytic | | |
| | | | behavior at | | |
| | | | varying pH | | |
| | | | levels | | |
| Time of | Hemolysis | Time not | Time not | 14 days (in | Hemolysis |
| Exposure | measured at | explicitly | explicitly | vivo), no | measured after |
| | 2, 4, 6, and | mentioned | mentioned | hemolysis | 20 min |
| | 24 h | | | observed | |
| Vehicle | PBS > | N/A | pH | N/A | N/A |
| Influence | Water > | | dependence: | | |
| | Ferulic acid | | hemolysis | | |
| | (FA) | | changes | | |
| | reduced | | dramatically | | |
| | hemolysis | | with pH | | |
| | | | shifts | | |
| Morphological | Significant | N/A | N/A | No RBC | No detailed |
| Changes in | membrane | | | damage | morphological |
| RBCs | damage | | | observed | study provided |
| | with <50 | | | | |
| | nm | | | | |
| | ZnONPs, | | | | |
| | mitigated by | | | | |
| | FA | | | | |
| Mechanism | Size- | Acidic, small | AgNP | N/A | Penetration of |
| of | dependent | molecular | aggregation | | NPs into weak |
| Hemolysis | ROS | weight proteins | at lower pH | | RBCs, |
| | generation, | preferentially | increases | | increasing |
| | osmotic | adsorbed onto | hemolysis; | | osmotic |
| | pressure | AgNPs, | bell-shaped | | pressure, |
| | increase, | influencing | hemolysis | | leading to cell |
| | and cell | hemolysis | curve | | lysis |
| | membrane | | | | |
| | damage | | | | |
| Protective | Ferulic acid | N/A | N/A | No protective | N/A |
| Mechanism | reduced | | | mechanism | |
| | hemolysis | | | needed due to | |
| | | | | minimal | |
| | | | | hemolysis | |
| | | | | observed | |
| Novelty | Demonstrated | Demonstrated | Unique | First to assess | Low hemolytic |
| and Impact | size- | that AgNPs | bell-shaped | XGO-PCL NPs | activity even at |
| on RBCs | dependent | with surface | hemolysis | for erythrocyte | high |
| | hemolytic | coatings caused | curve under | compatibility | concentrations; |
| | activity of | significant | varying pH | with less than 1 | high RBC |
| | ZnONPs; | hemolysis but | levels; first | % hemolysis, | resistance (80%), |
| | ferulic acid | did not affect | to report | indicating good | demonstrating |
| | reduced | platelet | such | blood safety | better |
| | hemolysis | aggregation or | behavior; | | biocompatibility |
| | | coagulation | hemolysis | | compared to |
| | | | higher for | | other NP types |
| | | | diabetic | | |
| | | | RBCs | | |

| | | Minimum dose causing | | Minimum dose causing | |
|---|---|---|---|---|---|
| Study | Nanoparticle type | minimum hemolysis | | maximum hemolysis | |
| Present | ZnPNPs | Maximum dose (5 | | Minimum dose (1 | |
| Study | | mg/mL) | | mg/mL) | |
| 1* | Silver NPs (PVP & citrate- | 10 | μg/mL | 40 | μg/mL |
| | coated) | | | | |
| 2* | Argovit ™ silver NPs | ~10 | μg/mL | ~40 | μg/mL |
| 3* | Silver NPs (AgNPs) | 50 | μg/mL | 200 | μg/mL |

| | | | |
|---|---|---|---|
| 4* | Xyloglucan-block- | <10 mg/kg/day | No significant hemolysis |
| | polycaprolactone copolymer | | (<1%) |
| | NPs | | |

1* refers to Huang, H and researchers, An Evaluation of Blood Compatibility of Silver Nanoparticles, Scientific Reports, 2016, 6, 25518;
2* refers to Luna-Vázquez-Gómez, R. et al., Bell Shape Curves of Hemolysis Induced by Silver Nanoparticles: Review and Experimental Assay, Nanomaterials (Basel), 2022, 12, 7, 1066;
3* refers to Preedia Babu, E. et al., Size-dependant uptake and hemolytic effect of zinc oxide nanoparticles on erythrocytes and biomedical potential of ZnO-ferulic acid conjugates, Scientific Reports, 2017, 7, 4203; and
4* refers to Mazzarino, L. et al., Nanoparticles Made From Xyloglucan-Block-Polycaprolactone Copolymers: Saftey Assessment for Drug Delivery, Toxicological Sciences, 2015, 147, 1, 104-115, which are incorporated herein by references in their entireties.

Aspects of the present disclosure provide synthesized dehydrated ZnPNPs, whereas other analyses utilized different zinc compound NPs, as listed in Table 10, which tabulates a comparison between this study and three other studies, focusing on the effects of NPs on RBCs hemolysis. No other research has examined the effects of ZnPNPs on vital organs, while the present disclosure focused on the aspect of using ZnPNPs. Regarding hemolysis, the present disclosure showed a lower hemolysis rate of 20% at high concentrations, particularly at concentrations ranging from 1 mg/mL to 5 mg/mL. Higher hemolytic activity at lower nanoparticle concentrations has been reported [Huang, H and researchers, An Evaluation of Blood Compatibility of Silver Nanoparticles, Scientific Reports, 2016, 6, 25518; Luna-Vázquez-Gómez, R. et al., Bell Shape Curves of Hemolysis Induced by Silver Nanoparticles: Review and Experimental Assay, Nanomaterials (Basel), 2022, 12, 7, 1066; and Preedia Babu, E. et al., Size-dependent uptake and hemolytic effect of zinc oxide nanoparticles on erythrocytes and biomedical potential of ZnO-ferulic acid conjugates, Scientific Reports, 2017, 7, 4203, which are incorporated herein by references in their entireties]. Xyloglucan block-polycaprolactone (XGO-PCL) NPs showed minimal hemolysis, even less than 1%, but did not focus on higher concentrations or ZnPNPs [Mazzarino, L. et al., Nanoparticles Made From Xyloglucan-Block-Polycaprolactone Copolymers: Safety Assessment for Drug Delivery, Toxicological Sciences, 2015, 147, 1, 104-115, which is incorporated herein by reference in its entirety]. Additionally, the present disclosure demonstrated that approximately 80% of RBCs resisted hemolysis, a result not observed in other analysis. Higher hemolysis rates and consistent RBC damage was seen across all concentrations of AgNPs. The ZnPNPs may affect weaker or older RBCs, indicating a selective mechanism of action. The present disclosure employed higher nanoparticle concentrations, including 1 mg/mL to 5 mg/mL, with relatively lower hemolytic effects.

DMSO alone exhibited a hemolytic effect on RBCs; however, the inclusion of ZnPNPs in the solution mitigates this effect. This finding may be used in applications involving ZnPNPs as a potential therapeutic agent, as it shows that ZnPNPs can protect RBCs from hemolysis, thus enhancing their biocompatibility.

Numerous modifications and variations of the present disclosure are possible in light of the above teachings. It is, therefore, to be understood that within the scope of the appended claims, the disclosure may be practiced otherwise than as specifically described herein.

1: A method of reducing hemolysis induced by dimethyl sulfoxide, comprising:
contacting a sample comprising red blood cells with an organic solvent composition comprising dimethyl sulfoxide and zinc phosphate nanoparticles in vitro at a temperature of 37° C.,
wherein the zinc phosphate nanoparticles are crystalline,
wherein the zinc phosphate nanoparticles are in the form of agglomerated, overlapping asymmetrical semi-spherical particles with an average size of 30 to 60 nm,
wherein the zinc phosphate nanoparticles are in the form of rectangular flakes,
wherein the zinc phosphate nanoparticles comprise 30 to 35 percent by weight (wt. %) oxygen, 12 to 18 wt. % phosphorous, and 50 to 55 wt. % zinc based on a total weight of the zinc phosphate nanoparticles, and
wherein the sample comprising red blood cells contacted with the organic solvent composition exhibits hemolysis of the red blood cells that is 4 to 5 times less than hemolysis of red blood cells in a sample contacted with an organic solvent composition comprising dimethyl sulfoxide without the zinc phosphate nanoparticles.

2: (canceled)

3: The method of claim 1, wherein the concentration of the dimethyl sulfoxide in the organic solvent composition is 0.1 to 1 percent by volume (vol. %) based on a total volume of the organic solvent composition.

4: The method of claim 1, wherein the sample comprising red blood cells has a free hemoglobin level of 0.05 to 0.25 g/L after the contacting.

5: The method of claim 1, wherein the zinc phosphate nanoparticles create a barrier on a surface of the red blood cells.

6: The method of claim 5, wherein the zinc phosphate nanoparticle barrier on the surface of the red blood cells does not allow the dimethyl sulfoxide to penetrate the red blood cells.

7: The method of claim 1, wherein 75% to 95% of the red blood cells in the sample are viable after the contacting.

8: The method of claim 1, further comprising:
sonicating the zinc phosphate nanoparticles in a polar solvent to form a solution;
contacting the solution with test cells,
wherein at least 15% of the test cells are viable at a zinc phosphate nanoparticle concentration of 1 mg/mL or less.

9: The method of claim 8, wherein the test cells are human embryonic kidney (HEK 293) cells.

10: The method of claim 8, wherein 105% to 115% of the test cells are viable at a solution concentration of 0.05 to 0.08 mg/mL.

11: The method of claim 8, wherein 110% to 115% of the test cells are viable at a solution concentration of 0.1 to 0.15 mg/mL.

12: The method of claim 8, wherein 100% to 110% of the test cells are viable at a solution concentration of 0.2 to 0.3 mg/mL.

13: The method of claim 8, wherein the zinc phosphate nanoparticles have a median lethal dose (LD₅₀) in rats of 2400 to 2600 mg/kg of body weight.

14: The method of claim 8, wherein the zinc phosphate nanoparticles have a median lethal dose (LD₅₀) in mice of 4800 to 5200 mg/kg of body weight.

15: The method of claim 8, wherein the contacting is from 5 to 50 days.

16: The method of claim 8, wherein the polar solvent comprises water, dimethyl sulfoxide, and a phosphate-buffered saline.

17: The method of claim 1, wherein the zinc phosphate nanoparticles have a polydispersity index of 0.8 to 0.9.

18: The method of claim 1, wherein the zinc phosphate nanoparticles have a zeta potential of −40 to −30 mV.

19: The method of claim 1, wherein the zinc phosphate nanoparticles comprise 33 to 34 wt. % oxygen, 14 to 15 wt. % phosphorous, and 52 to 53 wt. % zinc based on a total weight of the zinc phosphate nanoparticles.

20: The method of claim 1, wherein the zinc phosphate nanoparticles are made by a process, comprising:
dissolving a zinc salt in water and sonicating for 1 to 10 minutes to form a first solution;
dissolving a phosphate salt in water and sonicating for 1 to 20 minutes to form a second solution;
adding the second solution to the first solution to form a first mixture comprising particles and a liquid;
sonicating the first mixture for 20 to 60 minutes;
centrifuging the first mixture for 1 to 10 minutes;
decanting the liquid from the first mixture to separate the particles from the liquid;
washing the particles with water and centrifuging;
washing the particles with a polar solvent;
drying the particles;
calcinating the particles at a temperature of 700 to 900° C. for 1 to 5 hours to form calcinated particles; and
crushing the calcinated particles to form the zinc phosphate nanoparticles.

21: The method of claim 1, wherein the zinc phosphate nanoparticles are present in the organic solvent composition at a concentration of 0.0625 to 0.25 mg/mL.
